(19) United States
(12) Patent Application Publication
(10) Pub. No.: US 2016/0129044 A1
(43) Pub. Date: May 12, 2016

(54) USE OF MESOTHELIAL CELLS IN TISSUE BIOENGINEERING AND ARTIFICIAL TISSUES

(71) Applicants: FUNDACION PÚBLICA ANDALUZA PROGRESO Y SALUD, Sevilla (ES); INSTITUTO DE SALUD CARLOS III, Madrid (ES); VISSUM CORPORACIÓN, Alicante (ES); NEW BIOTECHNIC SA, Sevilla (ES)

(72) Inventors: Jorge Luis Alió y Sanz, Alicante (ES); Bernat Soria Escoms, Sevilla (ES); Christian Claude Lachaud, Sevilla (ES); Abdelkrim Hmadcha Afif, Sevilla (ES); Natalia Escacena Acosta, Sevilla (ES); Elena Quesada Fernández, Sevilla (ES); Felipe Soria, Alicante (ES)

(21) Appl. No.: 14/895,797

(22) PCT Filed: Jun. 5, 2014
(86) PCT No.: PCT/EP2014/061746

§ 371 Date: Dec. 3, 2015

(30) Foreign Application Priority Data
Jun. 5, 2013 (EP) 13382213.0

(51) Int. Cl. A61K 35/35; A61L 27/54; A61L 27/38; C12N 5/077; A61L 27/36

(52) U.S. Cl. CPC A61K 35/35; C12N 5/0653; A61L 27/3687; A61L 27/3804; A61L 27/54; C12N 2533/90; C12N 2500/30; A61L 2430/16

(57) ABSTRACT

Use of mesothelial cells and artificial tissues comprising mesothelial cells in regenerative medicine, wherein the mesothelial cells have been cultivated in a Mesothelial Retaining Phenotype Media (MRPM) containing a glucocorticoid, Culture media, pharmaceutical compositions and uses thereof.

### TECHNICAL FIELD OF THE INVENTION

The invention relates to the field of medical treatments, regenerative medicine and tissue engineering, and more specifically to the treatment of patients with mesothelial cells combined or not with biomaterials.

### BACKGROUND OF THE INVENTION

Tissue engineering is an interdisciplinary field which uses the concepts, principles and methods of material and life sciences to construct biological surrogates which are able to substitute the native tissue functions lost after disease or traumatic processes (Fuchs et al., 2001. Ann Thorac Surg 72:577-591; Shieh & Vacanti 2005. Surgery 137:1-7). Engineering of a successful tissue biomimetic relies on the election of an adequate cellular phenotype displaying functions that can substitute that of the damaged cells whilst keeping the ability to attach scaffolds and reproduce the biological and biophysical properties of the tissue.

In contrast with the amount of information available on skin tissue engineering (3873 works is related with the terms “skin tissue engineering” in Pubmed database) there is no information on the use of mesothelial cells regarding tissue engineering of an ex vivo biological substitute mimicking serous membranes. In the adult body, a serous membrane consists of a single layer of polygonal and flattened mesothelial cells attached to the surface of a thin layer of collagenous tissue. Serous membranes coat body cavities (pleural, pericardial and peritoneal) and the visceral organs where they are housed. The adult body contains several thin tissues in close interplay with fluids and sharing morphological and biochemical similarities with the mesothelium, for example the inner face of the cornea, bone joints, artificial organs, bladder, urethra, etc. The visceral adipose tissue mesothelium may therefore represent a source of autologous mesothelial cells which in combination with adequate scaffolds may allow the bioengineering of serosal membranes substitutes displaying structural and functional characteristics quite similar to other laminar tissues in close contact with fluids or recovering body cavities.

Mesothelium was first described by Bichat in 1827 as a fine layer of cells featuring characteristics of simple squamous epithelium. Although mesothelial cells display rather morphological and biochemical characteristics of epithelial cells, they however differ in their embryonic origin since mesothelial cells are derived from the mesoderm. This particularity confers mesothelial cells a unique phenotype as evidenced by their co-expression of squamous epithelial markers as well as specific mesenchymal markers (Mutsaers & Wilkosz 2007. Cancer Treat Res. 134: 1-19). A relevant main biological function displayed by mesothelial cells, is to secrete glycosaminoglycans and lubricants that remain entrapped between microvilli encountered at high density on their apical surface, thus providing a protective and slippery surface favouring an optimal sliding for the movements of visceral organs inside coelomic cavities, such as the beating heart or the expanding lungs (Odor 1954. Am J Anat. November; 95:433-65; Mutsaers 2002. Respirology. September; 7:171-91). Other reports evidence the central role of mesothelial cells in numerous intraserosal and submesothelial processes, including the transport of water and solutes, inflammation, host response, angiogenesis, tissue repair, and extracellular matrix remodeling (Mutsaers & Wilkosz. Cancer Treat Res 134: 1-19; Mutsaers 2002. Respirology 7: 171-191; Adam et al., 2002. J Cell Sci 115: 1383-1389). Particularly important for the invention are the presence and properties of membrane ion pumps on mesothelial cells (Park et al., 1998. Perit Dial Int 18: 402-409; Witowsky et al., 1997. Perit Dial Int 17: 186-193) and the capacity of mesothelial cells to secrete large amount of hyaluronan a critical compound regulating fluid homeostasis, tissue repair, inflammation status and response to infections (Honda et al., 1993. Biochem J 292 (Pt 2): 497-502; Oreopoulos 1998. Pent Dial Int 18: 382-386; Sitter et al., 2003. Perit Dial Int 23: 222-227). Hyaluronan also plays a critical role in the homeostasis of the anterior chamber, a fluid-filled space inside the eye between the iris and the cornea's innermost surface. Hyaluronan secreted by mesothelial cells may also improve significantly the lubrication of bone joints.

Cornea is a very complex tissue and is the best example of perfect natural engineering where the disposition of multilayers of collagen lamellaes, tightly compacted together and lacking blood vessels results in the formation of fully clear lens (DelMonte & Kim 2011. Cataract Refract Surg 37: 588-598). Even more, the corneal endothelium (a single layer of flat hexagonal cells tightly compacted and firmly adhered on the Descemet membrane plays an important role in regulating the state of corneal stromal hydration through the use of its potent Na+, K+-ATPase pump activity, which in combination with their tight junctions provide to this tissue a semipermeable layer allowing nutrients and other molecules to permeate from the aqueous humor. Corneal endotelial cells are highly active metabolically; they harbor numerous mitochondria to provide the sufficient energy required for the optimal function of membrane pumps. In adult humans, corneal endothelial cells are by high majority post mitotic (do not divide), this lack of regenerative capacities leads to the damaged or diseased corneal endothelium to a progressive loss of corneal endothelial cells density (Senoo & Joyce, 2000. Invest Ophthalmol Vis Sci. March; 41:660-7; Bourne 2003. Eye (Lond). November; 17:912-8.; Senoo et al., 2000. Invest Ophthalmol Vis Sci. September; 41:2930-5). As result, the lack of corneal endothelium leads to the disruption of the barrier to fluids flow from the aqueous humor to the stroma. Subsequently, this loss of function produces corneal edema, decreased corneal clarity, and finally results in a loss of visual acuity. Given these circumstances, the only effective treatment at present is provided by corneal transplantation to restore normal vision. In recent years, Descemet stripping automated endothelial keratoplasty (DSAEK) allowed a selective replacement of the diseases corneal endothelium achieving very good results (Mimura et al., 2013. Prog Retin Eye Res. 35: 1-17; Gorovoy 2006. Cornea September; 25:886-9). Despite the good results achieved, the use of this technique has been until now drastically limited by the low availability of cornea donors.

There are numerous attempts published on how to incorporate corneal endothelium as an alternative to a corneal transplantation: Endothelial Cells have been seeded onto chitosan-based membranes (Liang et al., 2011. J Mater Sci Mater Med. January; 22:175-83), Descemet's membrane (Insler & Lopez 1986. Curr Eye Res 5: 967-972; Insler & Lopez 1991. Invest Ophthalmol Vis Sci 32; 1828-1836; Insler & Lopez 1991. Cornea 10; 136-148; AboHLAChamat et al., 1999. Exp Eye Res 69: 547-553; Engelmann & Friedl 1989. In Vitro Cell Dev Biol 25: 1065-1072; Engelmann et al., 1999. Cornea 18: 199-206; Bohnke et al., 1999. Cornea 18: 207-213; Chen et al., 2001. Cornea 20: 731-737; Amano 2002. Nihon Ganka Gakkai Zasshi 106: 805-835; Amano 2003. Cornea 22: S66-74; Mimura et al., 2004. Exp Eye Res 79: 231-237), collagen matrix (Mimura et al., 2004. Invest. Ophthalmol. Vis. Sci, 45, 2992-2997), human corneal stromal discs (Honda et al., 2009. Arch. Ophthalmol. 127, 1321-1326; Choi et al., 2010. Biomaterials 31, 6738-6745), gelatin hydrogel discs (Lai et al., 2007. Transplantation 84, 1222-1232; Watanabe et al., 2011. Tissue Eng. Part A 17, 2213-2219), a cellular porcine corneal matrix (Ju et al., 2011. Indian J Med Res. June; 135:887-94). However, the lack of a source of corneal endothelial cells and the failure of adequate scaffolds to meet clinical demands such as biocompatibility, transparency, and mechanical strength have represented major problems of corneal tissue engineering.

The integrity of the synovial membrane is critical for the correct production and retention of synovial fluid which principally acts as lubricant to optimize the sliding of cartilage. As result of a trauma, synovitis or degenerative diseases such as rheumatoid arthritis, joint capsules may no longer maintain their normal integrity and functions, leading to a defective production or contention of the synovial fluid (Trofimova 1970. Vopr Revm 10: 55-61; Ostergaard et al., 2001. Ann Rheum Dis 60: 233-236).

Also, several cell types distinct have been already proposed to be used for the bioengineering of tracheal conduit biomimetic (He et al., 2012. Regen Med 7: 851-863). Similar to the trachea, the esophageous is a muscular large diameter conduit which may function is to allow the transient of the food from the oral cavity towards the stomach conduit. Other similar epithelium is the urothelial layer lining bladder lumen and urethra. There are many congenital conditions (mainly hypospadias and epispadias) or acquired conditions (traumatisms, stenosis, etc.) which affect its functional integrity and which require replacing it to a greater or lesser extent to re-establish its normal function (Baird et al., 2005, J Urol. 174:1421-4; Persichetti et al., 2006. Plast Reconstr Surg. 117:708-10).

It is necessary to develop new pharmaceutical compositions and artificial tissues to restore simple stratified squamous epithelium and serous membranes.

### SUMMARY OF THE INVENTION

In a first aspect, the invention relates to the use of mesothelial cells for preparing a medicament to partially or completely increase, restore or replace the functional activity of a diseased or damaged tissue or organ. In a preferred embodiment the mesothelial cells are cultivated in a suitable culture medium containing a glucocorticoid. In another preferred embodiment, the glucocorticoid is hydrocortisone. In another preferred embodiment, the diseased or damaged tissue is the endothelium. In another preferred embodiment, the diseased or damaged tissue is selected from the endothelium that lines the interior surface of blood vessels and lymphatic vessels, or the corneal endothelium. In another preferred embodiment, the diseased or damaged tissue is a serous membrane.

In a second aspect, the invention relates to an in vitro method (from hereinafter “method of the invention”) for preparing an artificial tissue comprising;
a) Sedding a support material with mesothelial cells, and
b) culturing the mesothelial cells in the support material of (a) in a suitable medium comprising a glucocorticoid, wherein preferably the suitable medium is the Mesothelial Retaining Phenotype Media (MRPM).

In a preferred embodiment of this second aspect of the invention, the suitable medium, preferably Mesothelial Retaining Phenotype Media (MRPM), contains hydrocortisone, in a more preferred embodiment the concentration of the hydrocortisone ranges between 0.1-100 μg/ml, and in a more preferred embodiment, the concentration of the hydrocortisone is about 1 μg/ml.

In another preferred embodiment, the mesothelial cells are derived from adipose tissue. In another preferred embodiment, the mesothelial cells are derived from autologous adipose tissue mesothelial cells (ATMCs).

In another preferred embodiment, the support material is from natural or synthetic origin. In a more preferred embodiment, the support material are threads with a monofilament or multifilament structure. In a yet more preferred embodiment, the support material is a silk nanofibers lamina. In another preferred embodiment, the support material is a suturing thread joined to a needle. In another preferred embodiment, the support material is a staple.

In a third aspect, the invention relates to an artificial tissue obtainable by the method of the invention (from hereinafter “artificial tissue of the invention”).

In a fourth aspect, the invention relates to the use of the artificial tissue of the invention for evaluating a pharmacological and/or chemical product.

In a fifth aspect, the invention relates to the artificial tissue of the invention for use in medicine or for use as a medicament, o alternatively to the use of the artificial tissue of the invention in medicine

In a sixth aspect, the invention relates to the artificial tissue of the invention for preparing a medicament to partially or completely increase, restore or replace the functional activity of a diseased or damaged tissue or organ, or alternatively to the use of the artificial tissue of the invention for preparing a medicament to partially or completely increase, restore or replace the functional activity of a diseased or damaged tissue or organ.

In a seventh aspect, the invention relates to a pharmaceutical composition comprising a mesothelial cell and/or the artificial tissue of the invention (from hereinafter “pharmaceutical composition of the invention”). In a preferred embodiment, the pharmaceutical composition of the invention further comprises a pharmaceutically acceptable carrier. In another preferred embodiment, the pharmaceutical composition of the invention comprises a further active ingredient.

In an eighth aspect the present invention relates to the use of a steroid hormone or a composition comprising a steroid hormone to avoid epithelial-mesenchymal transition (EMT). In a preferred embodiment, the steroid hormone is a gliucocorticoid, and more preferably is hydrocortisone.

In a ninth aspect, the present invention relates to a steroid hormone, or to a composition comprising a steroid hormone, for the prevention and treatment of fibrosis, and more preferably for the prevention and treatment of fibrosis of the peritoneal membrane.

In a tenth aspect, the present invention relates to a culture media (from hereinafter “culture media of the invention”), comprising a steroid hormone, preferably a glucocorticoid, and more preferably hydrocortisone. More preferably comprises a low concentration of serum with addition of culture supplements B27 and beta-mercaptoethanol (antioxidant).

In an eleventh aspect the invention relates to the use of the culture media of the invention to avoid epithelial-mesenchymal transition, and preferably to form cobblestone-like pavement in mesothelial cells cultured on plastic surface as well as on biological scaffold such is the case of anterior lens capsule (collagen lamina), as shown in the examples of the present invention.

### BRIEF DESCRIPTION OF THE FIGURES

FIG. 1. Isolation by trypsinization of murine adipose tissue mesothelial cells (ATMCs). (A) Left photograph shows representative aspect of mouse visceral adipose fat pads after surgical isolation. Right photograph shows representative aspect of adipose tissue mesothelial cells (ATMCs) released after trypsinization of adipose fat pads. (B) Characterization of ATMCs cultured for 48 hours in MRPM (Mesothelial Retaining Phenotype Media). Left image shows the typical mesothelial cobblestone-like morphology adopted by ATMCs cultures after 48 hours in MRPM. ATMCs displayed the typical epithelial intercellular expression (arrowhead) of the tight adhesion proteins β-catenin and ZO-1 (Zona Occludens-1). Nuclei are counterstained with Hoechst 33342.

FIG. 2. Adipose tissue mesothelial cells cultured in MRPM exhibit inhibition of Epithelial-to-Mesenchymal Transition (EMT). Left upper and lower photographs show phase contrast pictures of adipose tissue mesothelial cells (ATMCs) cultured for 48 hours in control media (media lacking hydrocortisone) and MRPM media formulation (Mesothelial Retaining Phenotype Media), that is control media containing 1 μg/ml hydrocortisone. ATMCs cultured in MRPM display cobblestone-like morphologies of epithelial cells. By contrast, ATMCs cultured in control media are more spindle shaped, morphology consistent with their initiation of EMT. Middle upper and lower images show F-actin immunostaining of ATMCs cultured in control media and MRPM. Consistent with EMT, ATMCs cultured in control media display increased F-actin expression and most importantly, display F-actin positive myofibrils in their cytoplasm, data consistent with their transition towards a mesenchymal smooth muscle-like phenotype. By contrast, ATMCs cultured in MRPM display F-actin positive ring-like staining, the typical pattern observed in epithelial cells. Right upper and lower images, show corresponding α-SMA staining, indicating that ATMCs cultured in control media exhibit α-SMA expression consistent with their initiation of EMT, whereas ATMCs cultured in MRPM display only very reduced expression of α-SMA, data confirming they did not undergo EMT. Nuclei are counterstained in blue with Hoechst 33342.

FIG. 3. Hydrocortisone (1 μg/ml) inhibits Serum- and EGF-induced EMT of adipose tissue mesothelial cells. (A) Comparative α-SMA immunofluorescence expression in ATMCs cultured for 3 days in basal media, basal media+1 μg/ml hydrocortisone (MRPM), MRPM+20 ng/ml EGF and basal media+20 ng/ml EGF. (B) Western blotting analysis, of the same experiment showing α-SMA expression in the distinct conditions relative to beta-actin expression. Each lane corresponds to 20 μg total protein.

FIG. 4. A glucocorticoid receptor antagonist (RU-486) counteracts hydrocortisone-inhibited EMT of ATMCs. (A) Comparative phase contrast images of ATMCs cultured for 5 days in MRPM and MRPM+10 μm RU-486. Not that ATMCs cultured in MRPM exhibit cobblestone-like monolayer, whereas ATMCs cultures performed in MRPM+10 μm display mixed epithelial and fibrobiastoid morphologies. Comparative α-SMA immunofluorescence staining indicates the presence of large fibrobiastoid α-SMA positive cells in the MRPM+10 μm RU-486 culture. (B) MetaMorph-based analysis of α-SMA immunofluorescence expression in ATMCs cultured for 5 days in MRPM and MRPM+10 μm RU-486. Values are representative of n=3 distinct cultures performed in the same conditions.

FIG. 5. Anterior Lens Capsule 12 hours post-seeding with Adipose tissue mesothelial cells. Upper left image shows 4× phase contrast picture of an Anterior Lens Capsule (HALC) seeded with mouse ATMCs and cultured for 12 hours in MRPM. Black arrowheads point to the external limit of HALC. Left inferior area of the picture shows plastic area with adhered ATMCs. Upper right image shows enlargement of the spot area drawn in upper left image. Black arrowheads point to the external limit of a HALC where numerous ATMCs accumulated next to its border. Lower image shows a higher magnification of HALO surface where many adherent ATMCs display typical polygonal morphology of mesothelial cells (arrow) spread onto HALC surface. At this time of HALC culture with ATMCs, only few cells displayed features of proliferation, identified by a rounded morphology (arrowhead).

FIG. 6. Anterior Lens Capsule 24 hours post-seeding with Adipose tissue mesothelial cells. Upper left image shows 4× phase contrast picture of an Anterior Lens Capsule (HALC) seeded with mouse ATMCs and cultured for 24 hours in MRPM. White arrows point to the external limit of HALO. Inferior area of the picture shows a plastic area with adhered ATMCs. Upper right image shows enlargement of HALO. White arrows point to the external limit of a HALO. Note that after 24 hours of MRPM culture, many ATMCs now display a more rounded morphology and are refringent (see arrows), morphological features indicating their active proliferation. Lower image shows a higher magnification of HALC surface where many adherent ATMCs display typical polygonal morphology of mesothelial cells (arrow) spread onto HALC surface. At this time of HALO culture with ATMCs, only few cells displayed features of proliferation, identified by a rounded morphology (arrowhead).

FIG. 7. Anterior Lens Capsule 72 hours post-seeding with Adipose tissue mesothelial cells. Upper left image shows 4× phase contrast picture of anAnterior Lens Capsule (HALO LAC) seeded with mouse ATMCs and cultured for 72 hours in MRPM. White arrowheads point to the external limit of HALO LAC. Upper right image shows enlargement of HALO LAC surface. White arrowheads point to the external limit of a HALC LAC. Note that after 72 hours of MRPM culture, ATMCs reached full confluence display a more rounded morphology and are refringent (see arrows), morphological features indicating their active proliferation. Lower left image shows a higher magnification of HALO surface where ATMCs display typical polygonal and cobblestone morphology of mesothelial cells. ATMCs reached full confluence and are tightly in contact to each other (arrow). Lower right image shows α-SMA and F-actin co-immunostaining fluorescence of ATMCs monolayer formed on top of HALC surface. Note that ATMCs retained F-actin expression restricted to their inner cytoplasmic membrane (arrowhead), a marker characteristic of the undifferentiated mesothelial cells. Consistent with their retention of a mesothelial phenotype, the cells display only limited expression of α-SMA expression which is detected as a diffuse cytoplasmic granular staining, feature indicating that ATMCs cultured in MRPM on top of HALC did not undergo EMT.

FIG. 8. TEM ultrastructural analysis of ATMCs cultured for 72 hours onto LACs. (A) Upper image shows a Transmission Electron Microscope (TEM) image of a thin transverse section through a 72 hours mesothelialized HALC. A total number of three ATMCs can be observed adhered on top of HALC basal membrane. Upper right image shows magnification of spot drawn in left image. ATMCs displayed typical apical membrane protrusions or microvilli on their apical surface (black arrows). ATMCs also displayed numerous mitochondria. Lower left image shows magnification of spot drawn in upper left image. Lower right image shows magnification of spot drawn in middle left image. Note how ATMCs display electron-dense tight junction complexes at apicolateral intercellular is contact. (B) Left image shows the representative aspect of ATMCs basal membrane in tight contact with HALC ATMCs basal membrane typically displayed numerous and regularly separated invaginations along all the portion of their basal membrane (black arrowheads). Intercalated between these invaginations were observed electron-dense plaques in contact with HALC surface, consistent with adhesion complexes. Right image shows a magnification of ATMCs basal membrane.

FIG. 9. Engineering of ATMCs sheets in vitro. (A) Left image shows phase contrast image of a monolayer of ATMCs cultured for 3 days in serum-free media containing B27 supplements and detached from the plastic surface. Detached ATMCs monolayer formed a rounded ultrathin sheet of ATMCs after full retraction of the cells. Right image shows magnification of ATMCs sheet after detachment and retraction. (B) F-actin immunostaining of ATMCs sheet. Left image shows a low power magnification of the F-actin stained mesothelial cells sheet. Right image shows a higher magnification allowing the appreciation of the high cellular density reached after cellular retraction. F-actin expression is very reduced and mostly localized to intercellular contacts.

FIG. 10. Schematic representation of the steps required to attain a full mesothelialization of silk nanofibers lamina. Seeding of silk nanofibers lamina with autologous adipose tissue mesothelial cells (ATMCs) and culture in MRPM leads to the adhesion, proliferation of ATMCs onto the lamina and reach full coverage establishing a compact mesothelial layer displaying contact inhibition growth.

FIG. 11. Schematic representation of binding of autologous mesothelial cells onto decellularized ECM or silk nanofibers lamina. The presence of Na⁺-Ca²⁺ ATP-ase ion pump on mesothelial cells allows fluid transport. Furthermore, mesothelial cells can synthesize and release hyaluronan in body cavities fluids.

FIG. 12. Schematic representation of the use of mesothelialized silk nanofibers lamina for the replacement of the synovial membrane. Bone joints with damaged synovium could be repaired with the use of synovial membrane biomimetic.

FIG. 13. Schematic representation of the use of mesothelialized silk nanofibers lamina for the replacement of serosal membrane. The serosal membrane devoid of mesothelium has exposed submesothelial tissue with submesothelial cells undergoing EMT towards fibroblasts, leading to peritoneal adhesion. Apposition of a serosal membrane biomimetic, i.e, silk nanofibers lamina harboring autologous adipose tissue mesothelial cells allows the restitution of a slippery non-adherent surface avoiding serosal membranes adhesion.

FIG. 14. Lining of the luminal layer of blood vessels grafts with a mesothelialized silk nanofibers lamina. Biologic or prosthetic vascular grafts are susceptible to be recovered by a monolayer of mesothelial cells to substitute the damaged endothelium.

FIG. 15. Isolation and establishment of MCECs cultures. Corneal endothelium stripping typically released large fragments of Descemet membrane (A) from which were detached large fragment of the corneal endothelium layer during the process of stripping. (B). (C) Adherent culture of MCECs clusters generated a compacted monolayer of MCECs exhibiting polygonal morphologies (see enlarged spot). (D) Morphology of MCECs at the end of their first subculture, with observable large clusters of tightly packaged MCECs. (E, F) Show representative aspect of MCECs morphologies at the end of their second and third subculture steps, respectively. Note how MCECs retained original cobblestone morphologies through sequential subculture steps.

FIG. 16. Quantitative RT-PCR analysis of corneal endothelium markers. Expression profile of COL4A2, COL8A2, SLC4A4, CAR2, Na+/K+-ATPase and N-cadherin genes was analyzed in whole corneas, stripped corneal endothelium, passage 3 subcultured MCECs (P3 MCECs), freshly isolated ATMCs (do ATMCs) and ATMCs cultured for 2 days into MRPM (d2 ATMCs). Stripped corneal endothelium isolated from six mice served as the calibrator sample (black bars). Gene expression was normalized against expression levels of YWHAZ (housekeeping gene). Results are mean fold change ±SD in mRNA expression relative to the calibrator sample (set as 1) calculated from three distinct isolation of corneas and three independent isolation and cultures of MCECs and ATMCs. Statistical significance (*P<0.05 and **P<0.03) was determined by using Student's t-test.

FIG. 17. Comparative immunofluorescence analysis of corneal endothelium markers expression patterns into cultured MCECs and ATMCs. Left panel shows representative immunoexpression levels of F-actin and of the corneal endothelium markers COL8A2, N-cadherin, ZO-1, β-catenin, Na+/K+-ATPase and SLC4A4 in mouse cornea for reference. Middle and right panel shows respective immunoexpression patterns obtained in subcultured MCECs (passage 3) and MRPM cultured ATMCs. Nuclei are counterstained in blue with Hoechst 33342.

### DETAILED DESCRIPTION OF THE INVENTION

The present invention shows that culture of mesothelial cells under specific conditions, maintains their original mesothelial phenotype and inhibits their epithelial-mesenchymal transition (EMT) in culture containing a low concentration of serum. Also shows that mesothelial cells can efficiently attach to different biomaterials, retaining both the capability to proliferate in a monolayer until it covers the whole area and to display contact inhibition of proliferation.

In this sense, the authors of the present invention have shown that the mesothelium adult visceral adipose tissue represents a valuable source to isolate autologous cells with capacity to substitute structurally and biochemically: i) the serosal wall of many organs and tissues; ii) damaged corneal endothelium; iii) cartilage and hyaluronan production; iv) mesothelial and endothelial cells in artificial urethra, trachea and other tissues and organs; v) endothelail cells in artificial blood vessels, etc.

Additionally, the authors of the present invention have developed a methodology to achieve a full mesothelialization of different biomaterials such as the decellularized basal membrane of anterior lens capsules, silk lamina, collagen, and other tissues from organs using mesothelial cells isolated from the visceral adipose tissue.

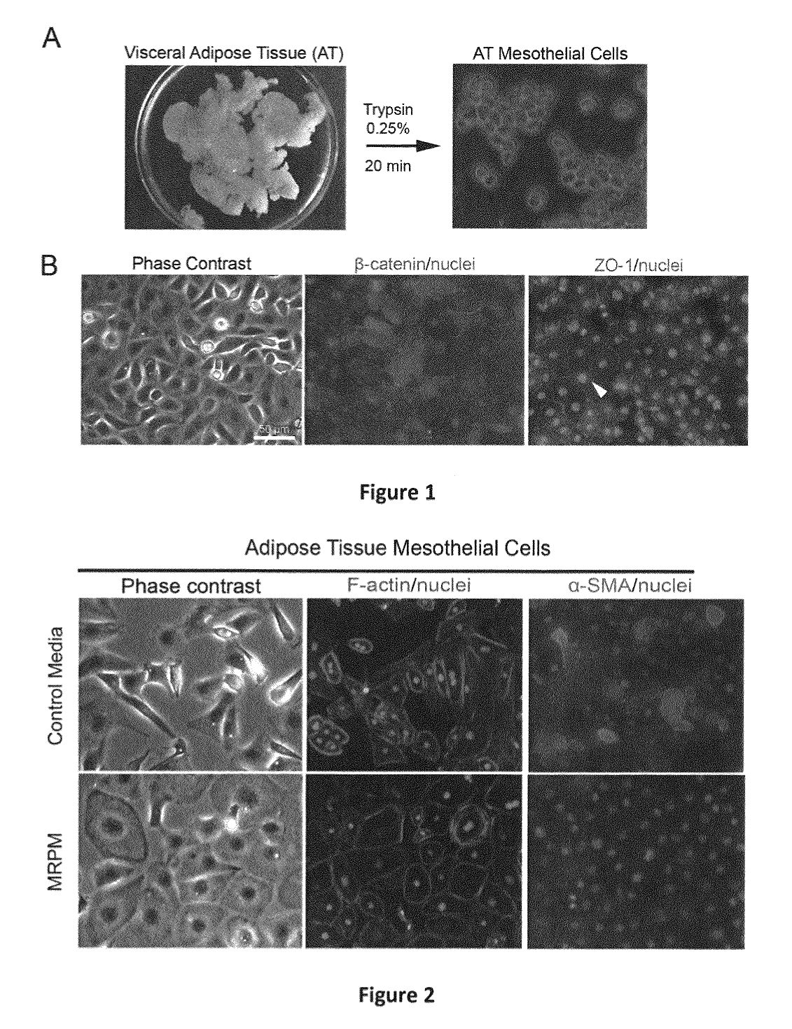

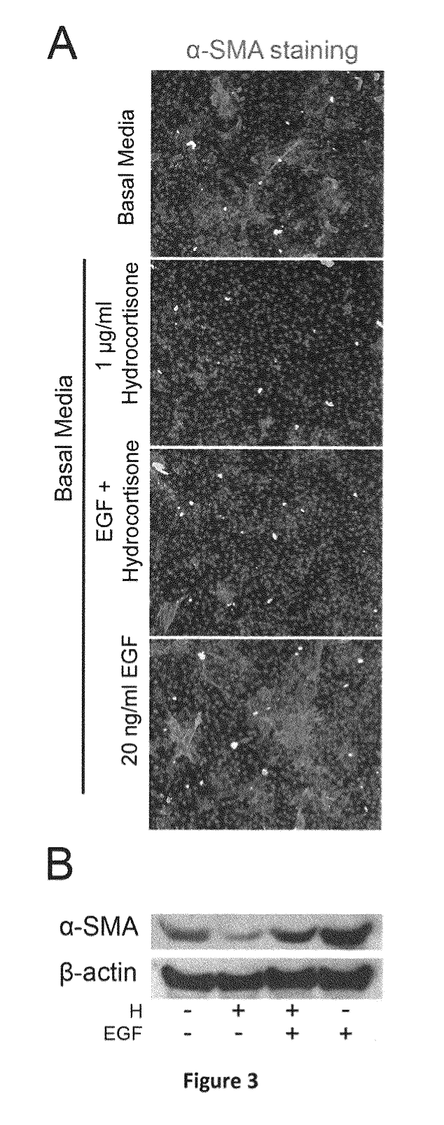

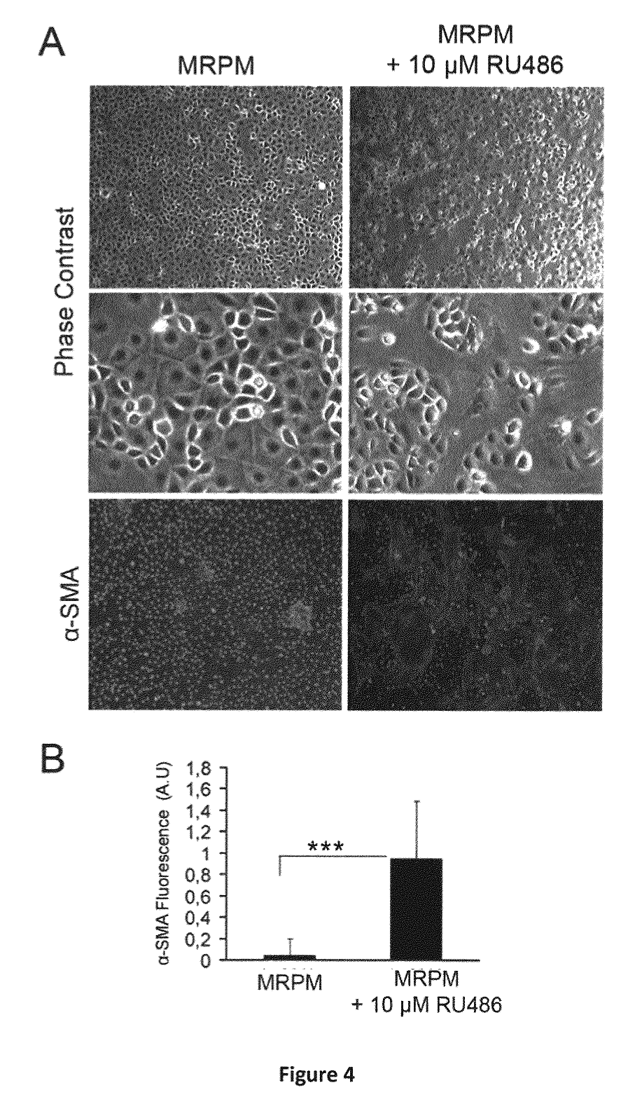

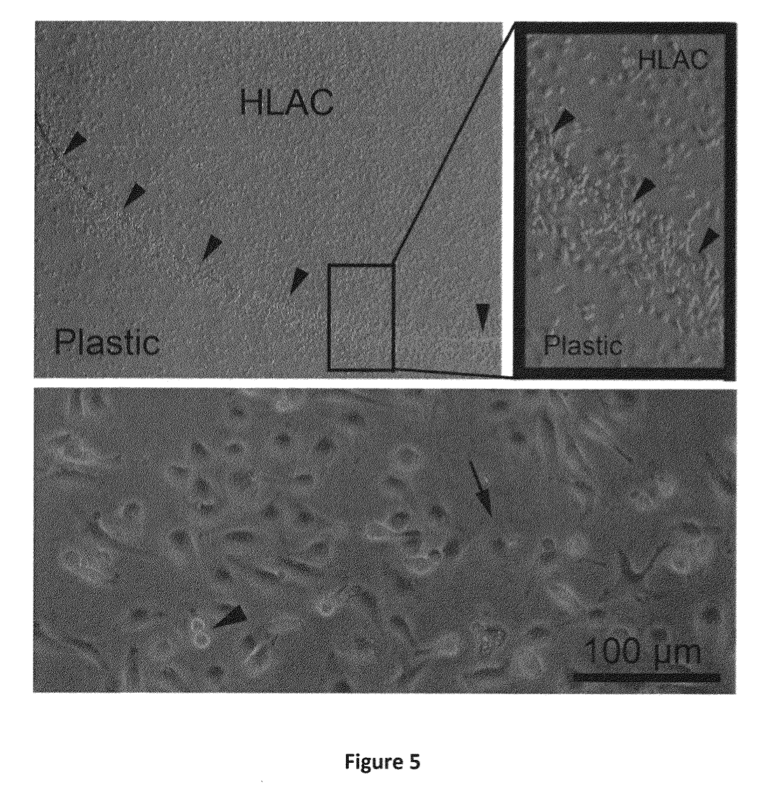

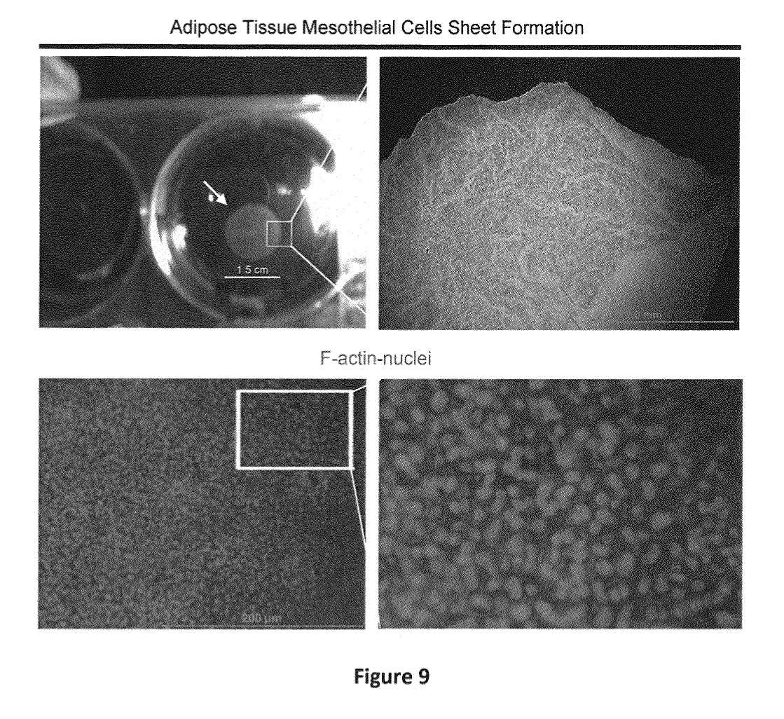

From the results shown in FIGS. 1-4, it can be clearly concluded that adipose tissue mesothelial cells cultured in a suitable medium such as MRPM (Mesothelial Retaining Phenotype Media) comprising a glucorticoid retain their original mesothelial phenotype in response to the medium formulation specially developed to reduce their epithelial-to-mesenchymal transition. From the results shown in FIGS. 5-8, it can be clearly concluded that adipose tissue mesothelial cells seeded onto Anterior Lens Capsules (HALC) adhere onto their surface, actively proliferate and recover their surface leading to a full coverage and generation of cobblestone-like monolayer exhibiting contact inhibition growth. FIG. 9, shows that ATMCs can be cultured in monolayer and can be detached to form a thin mesothelial membrane.

Thus, the present invention provides new uses for mesothelial cells to engineer artificial tissues and organs and to substitute endothelium, serous membranes and simple squamous epithelium, with the cells anchored to a support material as a basement membrane or not.

Thus, a first aspect of the invention refers to the use of mesothelial cells for preparing a medicament, or alternatively, to the mesothelial cells for use in medicine. Another aspect of the present invention refers to the use of mesothelial cells for preparing a medicament to partially or completely increase, restore or replace the functional activity of a diseased or damaged tissue or organ, or alternatively, to mesothelial cells to partially or completely increase, restore or replace the functional activity of a diseased or damaged tissue or organ. In a preferred embodiment the diseased or damaged tissue is the endothelium. In another preferred embodiment, the diseased or damaged tissue is selected from the endothelium that lines the interior surface of blood vessels and lymphatic vessels, or the corneal endothelium. In another preferred embodiment, the diseased or damaged tissue is a serous membrane. In another preferred embodiment, the diseased or damaged tissue is a simple squamous epithelium. In another preferred embodiment, the diseased or damaged tissue or organ is selected from the corneal endothelium, the synovial membrane, the inner tracheal layer, the esophageous epithelium and the bladder urothelium. In a preferred embodiment mesothelial cells are human mesothelial cells. In another preferred embodiment the mesothelial cells are derived from adipose tissue. In a more preferred embodiment, the mesothelial cells are cultivated in a suitable medium, preferably Mesothelial Retaining Phenotype Media (MRPM), containing a steroid hormone, more preferably a glucocorticoid, still more preferably hydrocortisone. In another preferred embodiment of the invention, the concentration of the hydrocortisone ranges from 0.1 to 100 μg/ml, more preferably from 0.5 to 50 μg/ml, and even more preferably the concentration of the hydrocortisone is about 1 μg/ml.

A further aspect of the invention refers to the use of human mesothelial cells, preferably human mesothelial cells from adipose tissue, cultivated in a suitable medium, preferably Mesothelial Retaining Phenotype Media (MRPM), containing a steroid hormone, more preferably a glucocorticoid, an even more preferably, hydrocortisone, for preparing a medicament to partially or completely increase, restore or replace the functional activity of a diseased or damaged tissue or organ. Preferably, the concentration of the hydrocortisone ranges from 0.1 to 100 μg/ml, more preferably from 0.5 to 50 μg/ml, and even more preferably the concentration of the hydrocortisone is about 1 μg/ml.

In a further embodiment, the cells may be implanted or injected into the patient together with a support material component. This may ensure that the cells remain at the appropriate location within the patient. In another preferred embodiment, the support material is from natural or synthetic origin. In a more preferred embodiment, the support material from natural origin is selected from the list consisting of: silk, decellularized bovine mesenteric serosal membranes, decellularized bovine pericardium and combinations thereof. In a yet more preferred embodiment, the support material are threads with a monofilament or multifilament structure, and in a particular embodiment, the support material is a silk nanofibers lamina.

Other examples of a support material include a collagen based support material, a fibrin based support material, a laminin based support material, a fibronectin based support material and artificial support materials. This list is provided by way of illustration only, and is not intended to be limiting. It will be clear to a person skilled in the art, that any combination of one or more matrix forming components may be used.

In a further embodiment, the cells may be contained within a microsphere. Within this embodiment, the cells may be encapsulated within the centre of the microsphere. Also within this embodiment, the cells may be embedded into the matrix material of the microsphere. The matrix material may include any suitable biodegradable polymer, including but not limited to alginates, Poly ethylene glycol (PLGA), fibrin and sericin and polyurethanes. This list is provided by way of example only, and is not intended to be limiting.

In a further embodiment, the cells may be adhered to a medical device intended for implantation. Examples of such medical devices include stents, pins, stitches, splits, pacemakers, prosthetic joints, artificial skin, and rods. This list is provided by way of illustration only, and is not intended to be limiting. It will be clear to a person skilled in the art, that the cells may be adhered to the medical device by a variety of methods. For example, the cells may be adhered to the medical device using fibrin, one or more members of the integrin family, one or more members of the cadherin family, one or more members of the selectin family, one or more cell adhesion molecules (CAMs), one or more of the immunoglobulin family and one or more artificial adherents. This list is provided by way of illustration only, and is not intended to be limiting. It will be clear to a person skilled in the art, that any combination of one or more adherents may be used.

Another aspect of the invention refers to an in vitro method (from hereinafter “method of the invention”), for preparing an artificial tissue comprising;
a) Sedding a support material with mesothelial cells, and
b) Culturing the mesothelial cells in the support material of (a) in a suitable medium or media, preferably Mesothelial Retaining Phenotype Media (MRPM), comprising a glucocorticoid.

Mesothelial cells are cells derived from mesothelium. Despite this, they rather display characteristics of simple squamous epithelial cells and as such they form a simple squamous epithelium lining the wall of body cavities and the visceral organs where they are housed. The main functions of mesothelial cells are to provide a slippery surface for the correct movements of visceral organs and also participate actively in transport of water and solutes, inflammation, host response, angiogenesis, tissue repair, and extracellular matrix remodeling.

As used herein “mesothelium” refers to a tissue solely endowed with physiological functions such as to maintain serosal integrity and inflammation and to secrete large amounts of “lubricants” to favors the correct sliding of opposite serosal layers.

The term “mesothelial cells” as used herein refers to cells isolated from mesothelium and expressing typical mesothelial cell markers including, but not limited to, Calretinin, Cytokeratins, Desmin, N-Cadherin, E-Cadherin, Keratin, Mesothelin, Vimentin, WT1 (Wilms' tumour susceptibility gene1), Zona Occludens 1 (ZO-1), β-catenin, Cytokeratin 18 (CK18), Cytokeratin 19 (CK19), CD44, CD29 (Integrin β1), CD54 (CAM-1) and Islet 1 (Isl1).

The mesothelial cells of step a) of the method of the invention are cultured and maintained in a suitable media comprising a glucocorticoid, preferably in a “mesothelial retaining phenotype media” (MRPM) to preserve their original phenotype, MRPM was formulated from a DMEM low glucose GlutaMax media (21885-05, Gibco) that was further supplemented with 2% heat inactivated FBS (Lonza), 1% B27 supplements (17504, Gibco), 1% penicillin-streptomycin (Gibco), 100 μM β-mercaptoethanol (31350-010, Gibco) and 1 μg/ml of hydrocortisone (Sigma Aldrich) as agent inhibiting epithelial-to-mesenchymal transition (EMT) of the mesothelial cells in culture.

Other types of suitable media capable of preserving the original phenotype of the cells, consist of a unique combination of DMEM low glucose GlutaMax media, heat inactivated FBS, B27 supplements, Penicillin-streptomycin, β-mercaptoethanol and Hydrocortisone.

In another preferred embodiment of the method of the invention, the suitable medium, preferably the Mesothelial Retaining Phenotype Media (MRPM), contains hydrocortisone.

In another preferred embodiment the concentration of the hydrocortisone ranges from 0.1 tp 100 μg/ml, more preferably from 0.5 to 50 μg/ml, and even more preferably the concentration of the hydrocortisone is about 1 μg/ml.

In another preferred embodiment the in vitro method of the invention comprises the following steps:
i) obtaining a composition comprising isolated mesothelial cells;
ii) culturing the isolated mesothelial cells from (i) in a suitable media, preferably the Mesothelial Retaining Phenotype Media (MRPM), containing a glucocorticoid;
iii) seeding the cultured isolated mesothelial cells from (ii) onto a support material, and
iv) culturing the isolated mesothelial cells on the support material of (iii) in a suitable medium, preferably the Mesothelial Retaining Phenotype Media (MRPM), containing a glucocorticoid.

In another preferred embodiment the in vitro method of the invention comprises the following steps:
i) adding a composition comprising trypsin to a sample of tissue comprising mesothelial cells,
ii) culturing the isolated mesothelial cells from (i) in a suitable media, preferably the Mesothelial Retaining Phenotype Media (MRPM), containing a glucocorticoid;
iii) seeding the cultured isolated mesothelial cells from (ii) onto a support material, and
iv) culturing the isolated mesothelial cells on the support material of (iii) in a suitable medium, preferably the Mesothelial Retaining Phenotype Media (MRPM), containing a glucocorticoid.

Preferably in this embodiment of the invention, the Mesothelial Retaining Phenotype Media (MRPM) contains hydrocortisone. In another preferred embodiment the concentration of the hydrocortisone ranges from 0.1 to 100 μg/ml, more preferably from 0.5 to 50 μg/ml, and even more preferably the concentration of the hydrocortisone is about 1 μg/ml. In a preferred embodiment mesothelial cells are human mesothelial cells. In another preferred embodiment, mesothelial cells are derived from adipose tissue, and more preferably from visceral adipose tissue. By “adipose tissue” is meant any visceral fat tissue. The visceral adipose tissue may be isolated from different anatomical origin, such as the visceral fat surrounding the heart or pericardial adipose tissue, or peritoneal visceral fat depots around kidney, mesenteric adipose tissue and from the omentum. If mesothelial cells are desired for autologous transplantation into a subject, the adipose tissue will be isolated from that subject.

“Adipose tissue-derived mesothelial cells” refers to mesothelial cells that originate from the visceral adipose tissue.

The mesothelial cells of the invention can be cells of autologous, allogeneic or xenogeneic origin. In a particular embodiment, said cells are of autologous origin and are isolated from adipose tissue of the subject to whom they will be administered, thus reducing potential complications associated with antigenic and/or immunogenic responses to said cells.

The term “culture” refers to any growth of cells, organisms, multicellular entities, or tissue in a medium. The term “culturing” refers to any method of achieving such growth, and may comprise multiple steps. The term “further culturing” refers to culturing a cell, organism, multicellular entity, or tissue to a certain stage of growth, then using another culturing method to bring said cell, organism, multicellular entity, or tissue to another stage of growth. A “cell culture” refers to a growth of cells in vitro. In such a culture, the cells proliferate, but they do not organize into tissue per se. A “tissue culture” refers to the maintenance or growth of tissue, e.g., explants of organ primordial or of an adult organ in vitro so as to preserve its architecture and function. A “monolayer culture” refers to a culture in which cells multiply in a suitable medium while mainly attached to each other and to a substrate. Furthermore, a “suspension culture” refers to a culture in which cells multiply while suspended in a suitable medium. Likewise, a “continuous flow culture” refers to the cultivation of cells or explants in a continuous flow of fresh medium to maintain cell growth, e.g. viability. The term “conditioned media” refers to the supernatant, e.g. free of the cultured cells/tissue, resulting after a period of time in contact with the cultured cells such that the media has been altered to include certain paracrine and/or autocrine factors produced by the cells and secreted into the culture. A “confluent culture” is a cell culture in which all the cells are in contact and thus the entire surface of the culture vessel is covered, and implies that the cells have also reached their maximum density, though confluence does not necessarily mean that division will cease or that the population will not increase in size. The term “culture medium” or “medium” is recognized in the art, and refers generally to any substance or preparation used for the cultivation of living cells. The term “medium”, as used in reference to a cell culture, includes the components of the environment surrounding the cells. Media may be solid, liquid, gaseous or a mixture of phases and materials. Media include liquid growth media as well as liquid media that do not sustain cell growth. Media also include gelatinous media such as agar, agarose, gelatin and collagen matrices. Exemplary gaseous media include the gaseous phase that cells growing on a petri dish or other solid or semisolid support are exposed to. The term “medium” also refers to material that is intended for use in a cell culture, even if it has not yet been contacted with cells. In other words, a nutrient rich liquid prepared for bacterial culture is a medium. Similarly, a powder mixture that when mixed with water or other liquid becomes suitable for cell culture may be termed a “powdered medium”. “Defined medium” refers to media that are made of chemically defined (usually purified) components. “Defined media” do not contain poorly characterized biological extracts such as yeast extract and beef broth. “Rich medium” includes media that are designed to support growth of most or all viable forms of a particular species. Rich media often include complex biological extracts. A “medium suitable for growth of a high density culture” is any medium that allows a cell culture to reach an OD600 of 3 or greater when other conditions (such as temperature and oxygen transfer rate) permit such growth. The term “basal medium” refers to a medium which promotes the growth of many types of microorganisms which do not require any special nutrient supplements. Most basal media generally comprise of four basic chemical groups: amino acids, carbohydrates, inorganic salts, and vitamins. A basal medium generally serves as the basis for a more complex medium, to which supplements such as serum, buffers, growth factors, lipids, and the like are added. Examples of basal media include, but are not limited to, Eagles Basal Medium, Minimum Essential Medium, Dulbecco's Modified Eagle's Medium, Medium 199, Nutrient Mixtures Ham's F-10 and Ham's F-12, Mc Coy's 5A, Dulbecco's MEM/F-I 2, RPMI 1640, and Iscove's Modified Dulbecco's Medium (IMDM).

The term “substantially pure”, with respect to adipose tissue-derived mesothelial cells populations, refers to a population of adipose tissue-derived mesothelial cells that is at least about 75%, preferably at least about 85%, more preferably at least about 90%, and most preferably at least about 95% pure, with respect to adipose tissue-derived stromal cells making up a total cell population. Recast, the term “substantially pure” refers to a population of adipose tissue-derived stromal cells of the present invention that contain fewer than about 20%, more preferably fewer than about 10%, most preferably fewer than about 5%, of lineage committed cells in the original unamplified and isolated population prior to subsequent culturing and amplification. “Support” as used herein refers to any device or material that may serve as a foundation or matrix for the growth of mesothelial cells, and more preferably for the growth of adipose tissue-derived mesothelial cells.

“Mesothelial Retaining Phenotype Media (MRPM)” is formulated from a DMEM low glucose GlutaMax media (21885-05, Gibco) supplemented with a low concentration (2%) of inactivated fetal bovine serum (Lonza), 1% B27 supplements (17504, Gibco), 1% penicillin-streptomycin (Gibco) and 100 μM of the anti oxidant β-mercaptoethanol (31350-010, Gibco). Additionally, the MRPM medium may further comprise a high concentration (1 μg/ml) of the glucocorticoid hydrocortisone (H0888, Sigma Aldrich) to inhibit EMT. An effective inhibition of the EMT of ATMCs is achieved with a hydrocortisone concentration ranging from 1 μg/ml to 100 ng/ml, preferably about 1 μg/ml.l

Unless otherwise defined there is no need to follow the combination order of the components defined in the precedent paragraph for MRPM

In another preferred embodiment of the in vitro method of the invention, the support material is from natural or synthetic origin. In a more preferred embodiment, the support material from natural origin is selected from the list consisting of: silk, decellularize bovine serosal membranes isolated from the mesentery, decellularized bovine pericardium and combinations thereof. In a yet more preferred embodiment, the support material are threads with a monofilament or multifilament structure, and in a particular embodiment, the support material is a silk nanofibers lamina.

Other examples of support material include a collagen based support material, a fibrin based support material, a laminin based support material, a fibronectin based support material and artificial support materials. This list is provided by way of illustration only, and is not intended to be limiting. It will be clear to a person skilled in the art, that any conventional or advanced biomaterial, orthopedic biomaterial or a combination of biomaterials.

In a more preferred embodiment, the support material are threads with a monofilament or multifilament structure.

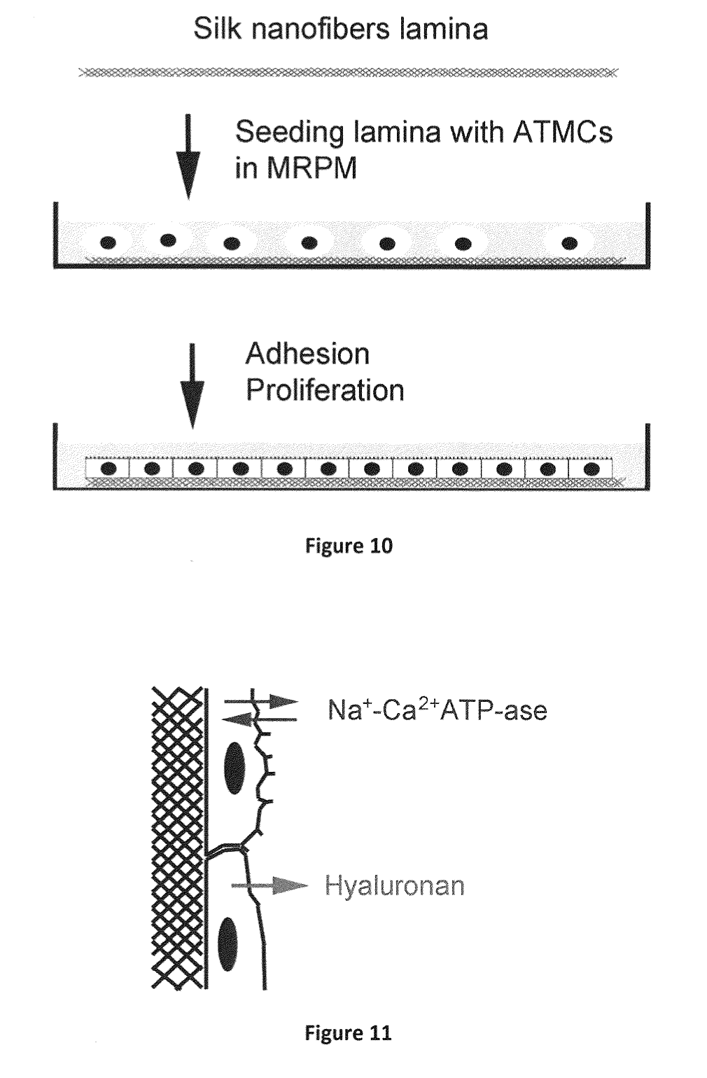

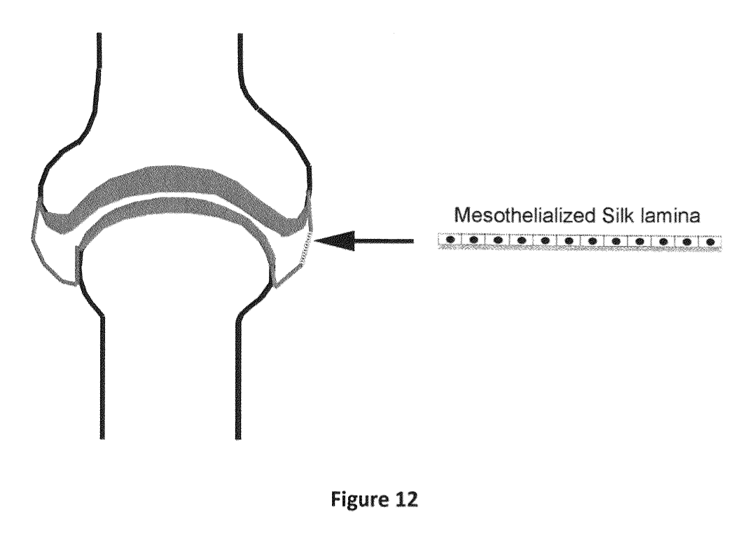

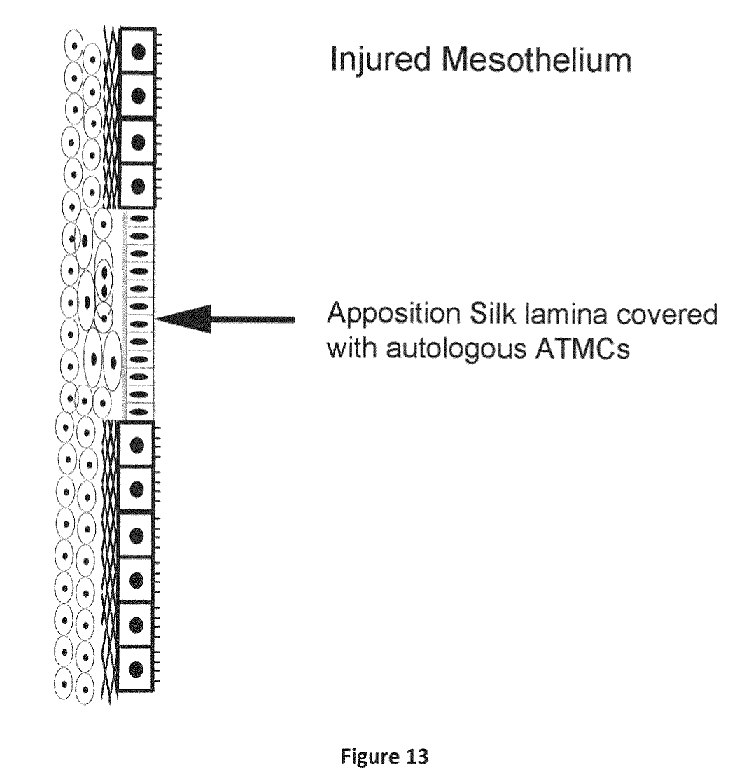

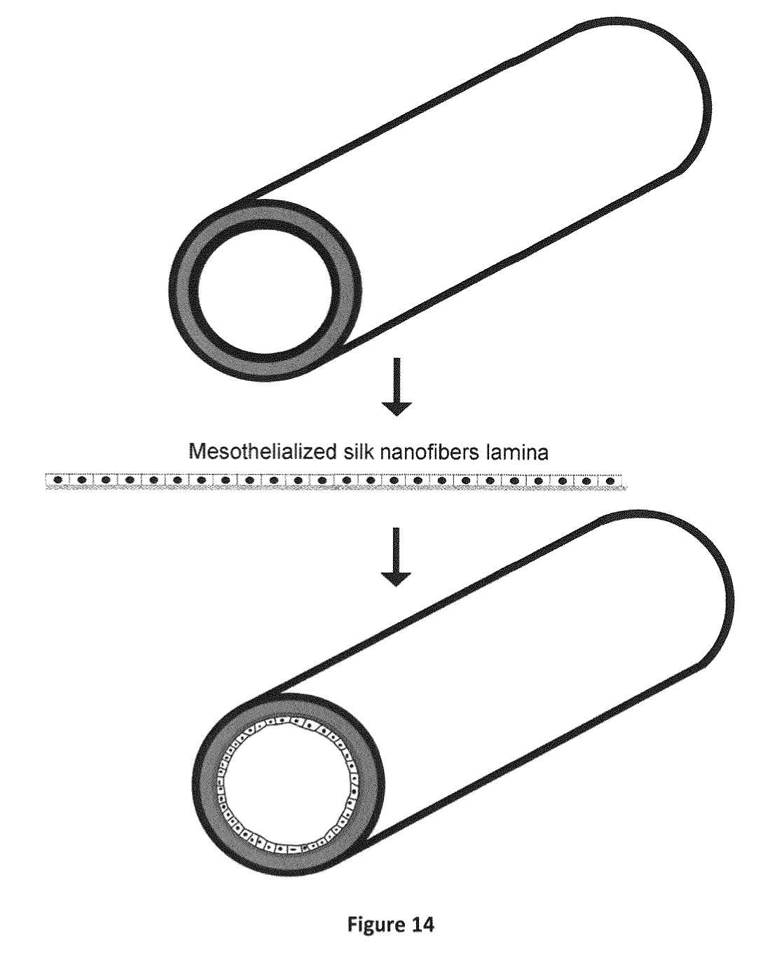

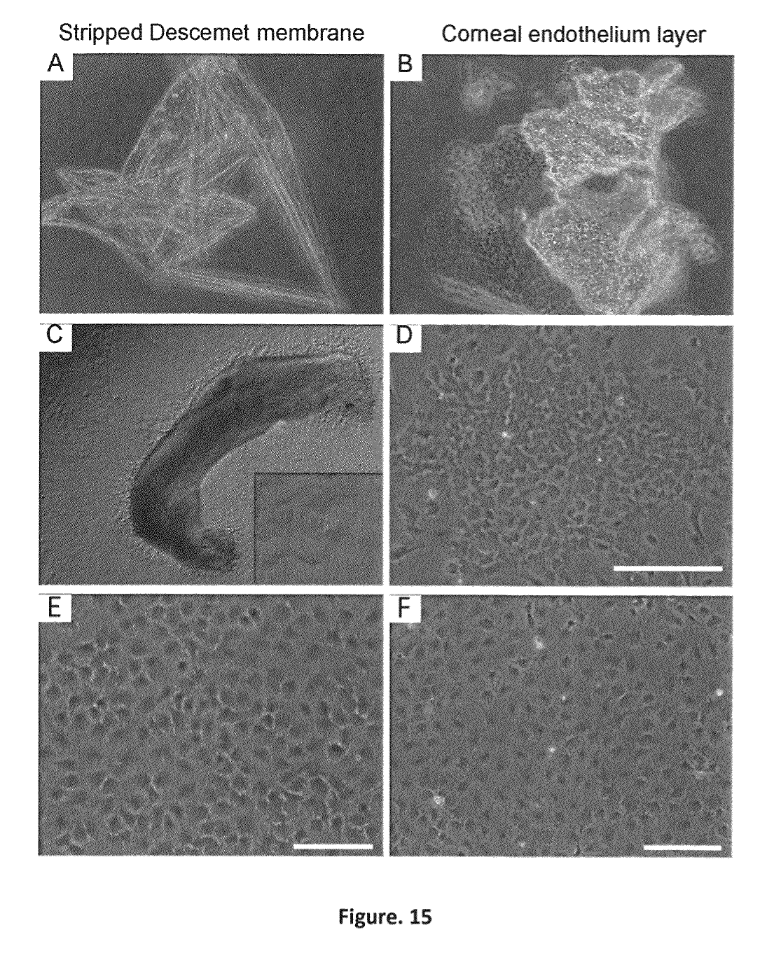

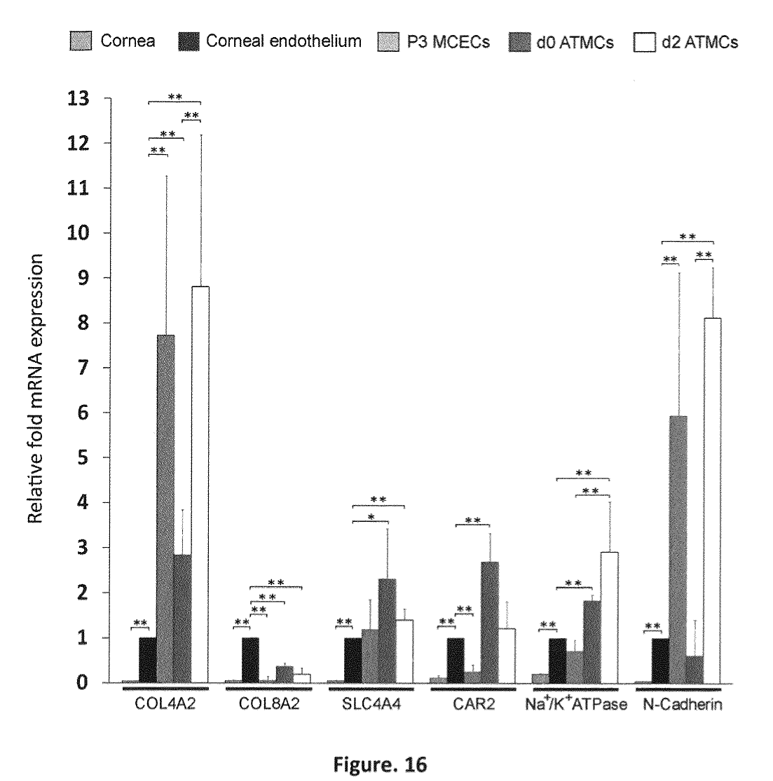

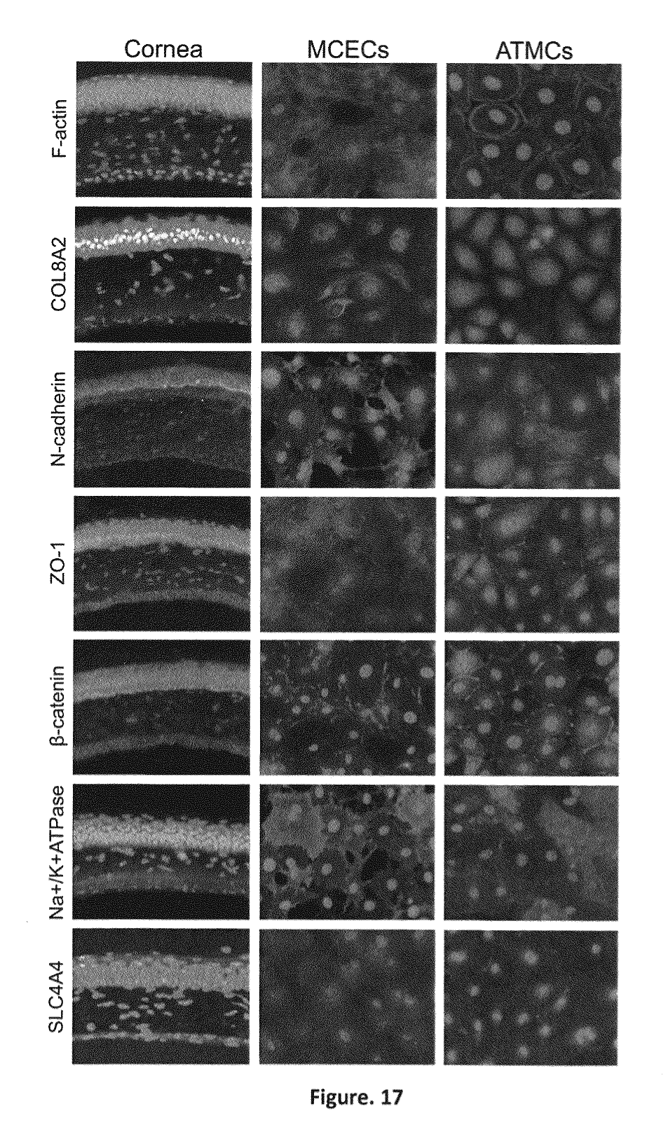

FIG. 10 shows a schematic representation of the steps required to attain a full mesothelialization of silk nanofibers lamina. Thus, in a yet more preferred embodiment, the support material is silk nanofibers lamina.

Traditionally, the suture has been the classic method for achieving rapid healing of tissues. Healing by primary intention suturing involves bringing the edges of the wound with the introduction of a suture to tissue using a metal needle attached to one end and making successive passes between both sides of the wound.

Additionally, the sutures are used in surgical practice to stop bleeding (hemostasis), as well as repair of organs and other structures of the human body. In some situations these sutures are delicate because of the difficulties of healing of tissues in which it sits.

One of the biggest drawbacks of the tissue suturing, is the fact that the needle diameter is larger than the thread, so that the insertion point of the needle will not be fully occupied by the latter, generating areas through which may be loss of fluids. This poor wound closure frequently causes post-operative complications, such as in the case of intestinal anastomoses performed in patients with carcinoma or diverticulosis, which performs a resection of the diseased intestinal and later joins the two healthy ends. In such patients, due to incomplete closure, they might lose stool with invasion of surrounding tissues, which will cause peritonitis, with the consequent risk to the patient's life. This risk is increased in patients with a reduced thickness of the intestinal wall, as in the case of inflammatory bowel disease. Mesothelial cells can be applied on the suture so that the opening is sealed generated by the passage of the thread through the fabric. Then, in another preferred embodiment, the support material is a suturing thread joined to a needle.

The term “suture” refers to a thread or fiber or other fastening material that can be used to sew a wound together.

The use of staples is an alternative to the classical method of suture. Allows primary closure of tissue in less time, reduces blood loss, reduces pollution and preserves blood flow. A limiting factor in the use of staples as a method of first intention cure is the need to have access to the top and bottom of the tissue to be joined. Also, due to the force exerted when inserting the staples may cause tearing of the tissue. Then, in another preferred embodiment, the support material is staples.

Artificial Tissue of the Invention

Another aspect of the invention refers to an artificial tissue obtainable by the in vitro method of the invention (from hereinafter “artificial tissue of the invention”).

In a preferred embodiment of this aspect of the invention, the artificial tissue of the invention is an endothelium replacing tissue or an artificial endothelium.

In another preferred embodiment of this aspect of the invention, the artificial tissue of the invention is a cornea replacing tissue or an artificial cornea.

In another preferred embodiment of this aspect of the invention, the artificial tissue is a serous membrane replacing tissue or an artificial serous membrane.

In another preferred embodiment of this aspect of the invention, the artificial tissue is a synovial membrane replacing tissue or an artificial synovial membrane.

In another preferred embodiment of this aspect of the invention, the artificial tissue is a trachea replacing tissue or an artificial trachea.

In another preferred embodiment of this aspect of the invention, the artificial tissue is an esophageous replacing tissue or an artificial esophageous.

In another preferred embodiment of this aspect of the invention, the artificial tissue is an orthopedic biomaterial.

In another preferred embodiment of this second aspect of the invention, the artificial tissue of the invention is a urethra replacing tissue or an artificial urethra.

The artificial tissue obtainable by the method of the invention can be cut into the desired size and/or can be provided in a suitable conformation for use.

Before use, the suitability of the artificial tissue of the invention for performing its function can be evaluated, for example, but not limited to, by means of any of the methods described in the examples of the present description.

Uses of the Artificial Tissue of the Invention in Pharmacological or Chemical Product Evaluation

The drugs and chemical products must be evaluated before their administration into test animals. To this respect, there are several reports and directives approved by the European Union which aim to restrict or even prohibit animal testing in the sector of cosmetic products (Directive 76/768/EEC of the European Council relating to the approximation of Member States' laws on cosmetic products), and the complete ban is expected to be in force in the next few years. The European Union supports all the measures the main objective of which is the wellbeing of the animals used for testing purposes and for achieving scientific replacement methods to reduce the number of animals used for testing to the minimum (Decision 1999/575/EEC of the Council, dated 23 Mar. 1998, relating to the conclusion by the Community of the European Convention for the protection of vertebrate animals used for experimental and other scientific purposes—Official Record L 222 of Aug. 24, 1999).

Therefore, another aspect of the invention relates to the use of the artificial tissue of the invention for evaluating a pharmacological and/or chemical product.

Therapeutic Uses of the Artificial Tissues of the Invention

An infectious, inflammatory, genetic or degenerative disease, physical or chemical damage, or blood flow interruption, can cause cell loss from a tissue or organ. This cell loss would lead to an alteration of the normal function of said tissue or organ; and consequently lead to the development of diseases or physical consequences reducing the person's quality of life. Therefore, attempting to regenerate and/or reestablish the normal function of said tissues or organs is important. The damaged tissue or organ can be replaced by a new tissue or organ which has been produced in the laboratory by means of tissue engineering techniques. The objective of tissue engineering is to construct artificial biological tissues and to use them for medical purposes to restore, replace or increase the functional activities of diseased tissues and organs. In addition, there are many advantages associated with the use of autologous cells or tissues in tissue engineering, is which include: (a) a significant reduction of the number of infections from the donor to the recipient by infectious agents; and (b) the absence of host immune graft rejection, therefore the patient does not need to undergo immunosuppressing treatment, side effects and problems associated with immunodepression being prevented.

Therefore, another aspect of the invention relates to the use of the artificial tissue of the invention to treat, in particular to partially or completely increase, restore or replace the functional activity, of a diseased or damaged tissue or organ. The artificial tissue of the invention can be used to treat, in particular to partially or completely increase, restore or replace the functional activity, of any diseased or damaged tissue or organ of a living organism. The tissue or organ can be for example, but not limited to, the urethra, or cornea. In a preferred embodiment the diseased or damaged tissue is the endothelium. In another preferred embodiment, the diseased or damaged tissue is selected from the endothelium that lines the interior surface of blood vessels and lymphatic vessels, or the corneal endothelium. In another preferred embodiment, the diseased or damaged tissue is a serous membrane. In another preferred embodiment, the diseased or damaged tissue is a simple squamous epithelium. In another preferred embodiment, the diseased or damaged tissue or organ is selected from the corneal endothelium, the synovial membrane, the inner tracheal layer, the esophageous epithelium and the bladder urothelium. In a preferred embodiment mesothelial cells are human mesothelial cells. In another preferred embodiment mesothelial cells and/or derived from adipose tissue. In a more preferred embodiment, the mesothelial cells have been cultivated in a Mesothelial Retaining Phenotype Media (MRPM) containing a steroid hormone, more preferably a glucocorticoid, an even more preferably, the glucocorticoid is the hydrocortisone. In another preferred embodiment the concentration of the hydrocortisone ranges between 0.1 and 100 μg/ml, and even more preferably the concentration of the hydrocortisone is about 1 μg/mi.

The tissue or the organ can be diseased or damaged as a result of a dysfunction, an injury or a disease, for example, but not limited to, an infectious disease, an inflammatory disease, a genetic disease or a degenerative disease; physical damage such as a traumatism or a surgical intervention, a chemical damage or blood flow interruption.

Another aspect of the present invention relates to the use of the artificial tissue of the invention for preparing a medicament, or alternatively, to the artificial tissue of the invention for use as a medicament.

Said medicament is a medicament for somatic cell therapy. “Somatic cell therapy” is understood as the use of living, autologous, allogenic or xenogenic somatic cells, the biological characteristic of which have been substantially altered as a result of their manipulation for obtaining a therapeutic, diagnostic or preventive effect through metabolic, pharmacological or immunological means. Among the medicaments for somatic cell therapy are, for example, but not limited to; cells manipulated to modify their immunological, metabolic or other type of functional properties in qualitative and quantitative aspects; sorted, selected and manipulated cells which are subsequently subjected to a manufacturing process for the purpose of obtaining the end product; cells manipulated and combined with non-cellular components (for example, biological or inert matrices or medical devices) performing the principle intended action in the finished product; autologous cell derivatives expressed ex vivo (in vitro) under specific culture conditions; cells which are genetically modified or are subjected to another type of manipulation to express homologous or non-homologous functional properties not expressed before.

Another aspect of the present invention relates to the artificial tissue of the invention for preparing a medicament to partially or completely increase, restore or replace the functional activity of a diseased or damaged tissue or organ, or alternatively to the use of the artificial tissue of the invention for preparing a medicament to partially or completely increase, restore or replace the functional activity of a diseased or damaged tissue or organ. In a preferred embodiment the diseased or damaged tissue is the endothelium. In another preferred embodiment, the diseased or damaged tissue is selected from the endothelium that lines the interior surface of blood vessels and lymphatic vessels, or the corneal endothelium. In another preferred embodiment, the diseased or damaged tissue is a serous membrane. In another preferred embodiment, the diseased or damaged tissue is a simple squamous epithelium. In another preferred embodiment, the diseased or damaged tissue or organ is selected from the corneal endothelium, the synovial membrane, the inner tracheal layer, the esophageous epithelium and the bladder urothelium. In a preferred embodiment mesothelial cells are human mesothelial cells. In another preferred embodiment mesothelial cells and/or derived from adipose tissue.

Use of Autologous Mesothelial Cells to Engineerer a Corneal Endothelium Biomimetic

The examples of the present invention show the use of mesothelial cells to restore the inner face of the cornea.

Use of Autologous Mesothelial Cells to Engineerer a Synovium Biomimetic

Bioengineering of synovial membrane mimetics with the use of autologous mesothelial cells could offer a new approach for repairing defective joint capsules. The secretion of hyaluronan by mesothelial cells would have application for substituting the synovial lining cells of joint capsules, that secrete hyaluronan in the synovial fluid, compound that exert a potent anti inflammatory action and inhibit the production of matrix metalloproteinase-1 (MMP-1) (Shimizu et al., 2003. J Rheumatol 30: 1164-1172; Scott et al., 2000. Microvasc Res 59: 345-353).

Use of Autologous Mesothelial Cells to Engineerer Tracheal Epithelium Biomimetic

The inner layer covering of the trachea is a cilia-lined mucus membrane (Smith et al., 2008. Respir Physiol Neurobiol 163: 178-188). Several cell types distinct to mesothelial cells have been already proposed to be used for the bioengineering of tracheal conduit biomimetic (He et al., 2012. Regen Med 7: 851-863).

Use of Autologous Mesothelial Cells to Engineerer an Esophageous Epithelium Biomimetic

Similar to the trachea, the esophageous is a muscular large diameter conduit which main function is to allow the transient of the food from the oral cavity towards the stomach conduit. The esophageous is composed of four main layers, the adventitia, muscularis propia, submucosa and mucosa. The luminal mucosa layer is a stratified squamous epithelium lining the lumen. The esophagous contains numerous glands under the epithelium which secrete large amounts of lubricants recovering the luminal surface with the functions to protect the outermost epithelium layer and to improve the sliding of food towards the stomachal conduit.

Use of Autologous Mesothelial Cells to Engineerer a Bladder Urothelium Biomimetic

Other epithelium susceptible to be replaced with autologous mesothelial cells is the urothelial layer lining bladder lumen and urethra. The urothelium is a transitional epithelium consisting of 3-5 layers, increasing in complexity from the basal laminae towards the luminal surface. Autologous mesothelial cells deposited in the form multilayers on top of a laminar scaffold (i.e, lamina of silk nanofibers) allow the generation of biological mimetic for the bladder urothelium.

Use of Autologous Mesothelial Cells to Engineerer Serosal Membranes Biomimetics

Peritoneal adhesions resulting from peritoneal surgery or unintentional tissue injury often result from a loss of the mesothelial cells layers, leading to the adhesion between opposite submesothelial layers. They represent a significant health problem with major implications on quality of life and health care expenses (Rizzo et al., 2010. Immunopharmacol Immunotoxicol 32: 481-494; Schnuriger et al., 2011. Am J Surg 201: 111-121). The isolation, propagation and use of autologous mesothelial cells from the visceral adipose in combination with fine laminar scaffolds reproducing the submesothelial matrix allow the generation of serosal membranes biomimetics with the application to replace areas of damaged mesothelium. The substitution of damaged mesothelium areas restore it slippery surface. The engraftment of bioengineered mesothelial membranes restitutes a slippery and non adherent surface.

Use of Autologous Mesothelial Cells to Bioengineerer Serosal Membranes for Artificial Visceral Organs.

Bioengineering of artificial organs (i.e, heart, bladder and liver) is under current research, mainly to find biological or artificial scaffolds with the ideal conformation, strength and biocompatibility for the seeding, homing and differentiation of the different cellular phenotype components proper of each organ. Because visceral organs are always recovered by a serosal membrane, autologous mesothelial cells isolated from the visceral adipose tissue represent a valuable source of mesothelial cells with in combination with the adequate laminar scaffold, in example cultured onto silk nanofibers lamina, may represent a mesothelium biomimetic to cover their entire surface.

Biomaterials for Use in Combination with Mesothelial Cells to Bioengineerer Serosal Membranes Biomimetics.

Silk

In the present invention is show how to bioengineer silk sheets and use them as a supportive scaffold in combination with autologous mesothelial cells to build a biomimetic of serosal membranes. The silk of the mulberry silkworm Bombyx mori is primarily composed by two proteins, sericin and fibroin (Chen et al., 2012. Acta Biomater 8: 2620-2627.), being its structural core composed by fibroin. This fiber is embedded by an external sticky layer of tightly compacted sericin. The silk fiber display extraordinary mechanical strength and biophysical properties, making of this material an ideal component for the generation of laminar scaffold. Furthermore, the silk is a biological material that is degraded into the body relatively slowly over time. Electrospinning of silk fibroin solution allows the generation of nanofibers which deposition in multilayers allow the generation of a lamina of several micrometers of thickness. The silk nanofibers lamina mimics in most extent the submesothelial connective tissue matrix, which is mainly composed by thick collagen fibers, the amorphous component of elastic fibers, a matrix that offers a supportive layer for the anchorage of mesothelial cells.

Decellularized Bovine Serosal and Epicardial Mesothelium

Alternative to use of bioartificial laminar matrix such is the electrospun silk nanofibers lamina, is possible use decellularize bovine serosal membranes isolated from the mesentery. The mesentery contains large surface of transparent serosal membranes, consisting of two layers of mesothelial cells adhered onto a matrix mainly composed by collagen fibers. The decellularization of these serosal membranes through the use of detergent (Triton-X 100) and enzymatic digestion (trypsin) would achieve the effective release of the mesothelial cells layers and the generation of a fine meshwork of collagen fibers to seed autologous mesothelial cells, in order to reconstruct a serosal membrane biomimetic.

Decellularized Bovine Pericardium

Other source of extracellular matrix (ECM) offering a supportive scaffold for adhesion and growth of mesothelial cells is the pericardium, which is thicker that the mesenteric serosal membrane ECM. Decellularization of the bovine pericardium with the use of detergents and enzymes and the posterior cross-linking of the resulting ECM has been already achieved (Oswal et al., 2007. J Heart Valve Dis 16: 165-174; Neethling et al., 2008. J Heart Valve Dis 17; 456-463; Yang et al., 2009. J Biomed Mater Res B Appl Biomater 91: 354-361) and used as supporting scaffold in cardiovascular surgery, principally to reconstruct heart valves (Yang et al., 2012. J Biomed Mater Res B Appl Biomater 100: 1654-1661), Nonetheless, the use of decellularized pericardium in combination with autologous mesothelial cells has still not been achieved. On this basis, the generation of large area of decellularized pericardium allows the construction conduit scaffold which luminal recovering with autologous mesothelial cells represent the primary structure required to bioengineer trachea and esophageous biosubstitute.

Pharmaceutical Composition

Another aspect of the invention relates to a pharmaceutical composition comprising a mesothelial cell of the invention and/or the artificial tissue of the invention.

A preferred embodiment of this aspect of the invention relates to a pharmaceutical composition comprising a mesothelial cell of the invention and/or the artificial tissue of the invention for use in somatic cell therapy.

A more preferred embodiment of this aspect of the invention relates to a pharmaceutical composition comprising a mesothelial cell of the invention and/or the artificial tissue of the invention to partially or completely increase, restore or replace the functional activity of a tissue or organ.

A preferred embodiment of this aspect of the invention relates to a pharmaceutical composition comprising a mesothelial cell of the invention and/or the artificial tissue of the invention to partially or completely increase, restore or replace the functional activity of a diseased or damaged tissue or organ as a result of an infectious disease, inflammatory disease, genetic disease or degenerative disease, physical or chemical damage or blood flow interruption. In a preferred embodiment the diseased or damaged tissue is the endothelium. In another preferred embodiment, the diseased or damaged tissue is selected from the endothelium that lines the interior surface of blood vessels and lymphatic vessels, or the corneal endothelium. In another preferred embodiment, the diseased or damaged tissue is a serous membrane. In another preferred embodiment, the diseased or damaged tissue is a simple squamous epithelium. In another preferred embodiment, the diseased or damaged tissue or organ is selected from the corneal endothelium, the synovial membrane, the inner tracheal layer, the esophageous epithelium and the bladder urothelium. In a more preferred embodiment, the mesothelial cells are cultivated in a suitable medium, preferably the Mesothelial Retaining Phenotype Media (MRPM), containing a steroid hormone, more preferably a glucocorticoid, an even more preferably, hydrocortisone. In another preferred embodiment the concentration of the hydrocortisone ranges from 0.1 to 100 μg/ml, and even more preferably the concentration of the hydrocortisone is about 1 μg/ml.

In a preferred embodiment mesothelial cells are human mesothelial cells. In another preferred embodiment mesothelial cells and/or derived from adipose tissue.

In a preferred embodiment of this aspect of the invention, the pharmaceutical composition comprises a mesothelial cell of the invention and/or the artificial tissue of the invention and also a pharmaceutically acceptable carrier. In another preferred embodiment of this aspect of the invention, the pharmaceutical composition comprises the artificial tissue of the invention and also another active ingredient. In a preferred embodiment of this aspect of the invention, the pharmaceutical composition comprises a mesothelial cell of the invention and/or the artificial tissue of the invention and also another active ingredient together with a pharmaceutically acceptable carrier.

As used herein, the term “active ingredient”, “active substance”, “pharmaceutically active substance”, “active ingredient” or “pharmaceutically active ingredient” means any component which potentially provides a pharmacological activity or another different effect in diagnosing, curing, mitigating, treating, or preventing a disease, or which affects the structure or function of the human body or body of other animals. Examples of active ingredients of biological origin include growth factors, hormones, and cytokines. A variety of therapeutic agents are known in the art and may be identified by their effects. Certain therapeutic agents are capable of regulating cell proliferation and differentiation. Examples include chemotherapeutic nucleotides, drugs, hormones, non-specific (non-antibody) proteins, oligonucleotides (e.g., antisense oligonucleotides that bind to a target nucleic acid sequence (e.g., mRNA sequence)), peptides, and peptidomimetics.

The pharmaceutical compositions of the present invention can be used in a treatment method in an isolated manner or together with other pharmaceutical compounds.

The term “pharmaceutically acceptable excipient” as used here refers to the fact that it must be approved by a regulatory agency of the federal government or a national government or one listed in the United States Pharmacopoeia or the European Pharmacopoeia, or some other pharmacopoeia generally recognized for use in animals and in humans. The term “vehicle” relates to a diluent, excipient, carrier or adjuvant with which the stem cells, progenitor cells or differentiated cells of the invention, the immortalized cells of the invention, as well as the cells of the cell population of the invention, must be administered; obviously, said vehicle must be compatible with the cells. Illustrative, non-limiting examples of said vehicle include any physiologically compatible vehicle, for example isotonic solutions (e.g. sterile saline solution (0.9% NaCl), phosphate-buffered saline solution (PBS), Ringer-lactate solution, etc.), optionally supplemented with serum, preferably with autologous serum; culture media (e.g. DMEM, RPMI, McCoy, etc.); or, preferably, a solid, semisolid, gelatinous or viscous support medium, such as collagen, collagen-glycosamine-glycan, fibrin, polyvinyl chloride, poly-amino acids, such as polylysine, or polyornithine, hydrogels, agarose, dextran sulphate silicone. Moreover, if desired, the support medium can, in special embodiments, contain growth factors or other agents. If the support is solid, semisolid, or gelatinous, the cells can be introduced in a liquid phase of the vehicle that is treated subsequently so that it is converted into a more solid phase. In some embodiments of the invention in which the vehicle has a solid structure, said vehicle can be configured according to the form of the lesion.

The pharmaceutical composition of the invention can, if desired, also contain, when necessary, additives for increasing and/or controlling the desired therapeutic effect of the cells, e.g. buffering agents, surface-active agents, preservatives, etc. The pharmaceutically acceptable carrier may comprise a cell culture medium which supports the cells' viability. The medium will generally be serum-free in order to avoid provoking an immune response in the recipient. The carrier will generally be buffered and/or pyrogen free. Also, for stabilizing the cellular suspension, it is possible to add chelating agents of metals. The stability of the cells in the liquid medium of the pharmaceutical composition of the invention can be improved by adding additional substances, such as, for example, aspartic acid, glutamic acid, etc. Said pharmaceutically acceptable substances that can be used in the pharmaceutical composition of the invention are generally known by a person skilled in the art and are normally used in the production of cellular compositions. Examples of suitable pharmaceutical vehicles are described in “Remington's Pharmaceutical Sciences” by E. W. Martin. Additional information on said vehicles can be found in any manual of pharmaceutical technology (that is, galenical pharmacy).

The pharmaceutical composition of the invention will be administered in a suitable pharmaceutical form of administration. For this, the pharmaceutical composition of the invention will be formulated according to the chosen form of administration. The formulation will be adapted to the method of administration. In a special embodiment, the pharmaceutical composition is prepared in a liquid, solid or semisolid dosage form, e.g. in the form of suspension, in order to be administered by implanting, injection or infusion to the subject needing treatment. Illustrative, non-limiting examples include formulation of the pharmaceutical composition of the invention in a sterile suspension with a pharmaceutically acceptable excipient, e.g. an isotonic solution, for example, phosphate-buffered saline solution (PBS), or any other suitable, pharmaceutically acceptable vehicle, for administration to a subject parenterally, although other routes of administration can also be used.

The administration of the pharmaceutical composition of the invention to the subject who needs it will be carried out using conventional means. In a particular embodiment, said pharmaceutical composition of the invention can be administered to the subject parenterally using suitable devices such as syringes, catheters, trocars, cannulas, etc. In all cases, the pharmaceutical composition of the invention will be administered using equipment, apparatus and devices suitable for the administration of cellular compositions and known by a person skilled in the art. In another embodiment, direct administration of the pharmaceutical composition of the invention to the site that is intended to benefit may be advantageous. In this method, direct administration of the pharmaceutical composition of the invention to the desired organ or tissue can be achieved by direct administration (e.g. by injection, etc.) on the external surface of the affected organ or tissue by inserting a suitable device, e.g. a suitable cannula, by infusion (including reverse flow mechanisms) or by other means described in this patent or known in the art.

The pharmaceutical composition of the invention can be stored until the moment of its application by the conventional methods known by a person skilled in the art. For short-term storage (less than 6 hours), the pharmaceutical composition of the invention can be stored at or below room temperature in a sealed container, supplemented or not with a nutrient solution. Medium-term storage (less than 48 hours) is preferably carried out at 2-8[deg.]C, and the pharmaceutical composition of the invention includes, in addition, an iso-osmotic, buffered solution in a container made of or lined with a material that prevents cellular adhesion. Longer-term storage is preferably carried out by means of suitable cryopreservation and storage in conditions that promote the retention of cellular function.

In a concrete embodiment, the pharmaceutical composition of the invention can be used in combination therapy. Said additional medicinal products can form part of the same pharmaceutical composition or can, alternatively, be supplied in the form of a separate composition for simultaneous or successive (sequential in time) administration relative to the administration of the pharmaceutical composition of the invention.

Culture Media Comprising Steroid Hormones and Uses

Another aspect of the invention relates to the use of a steroid hormone to avoid epithelial-mesenchymal transition. In a preferred embodiment, the steroid hormone is a gliucocorticoid, and more preferably is the hydrocortisone.

Another aspect of the invention relates to the use of a composition comprising a steroid hormone to avoid epithelial-mesenchymal transition. In a preferred embodiment, the steroid hormone is a glucocorticoid, and more preferably is the hydrocortisone.

Another aspect of the invention relates to an steroid hormone, or to the composition comprising a steroid hormone, for the prevention and treatment of fibrosis, and more preferably for the prevention and treatment of fibrosis of the peritoneal membrane, or alternatively to the use of a steroid hormone, or a composition comprising a steroid hormone, for the manufacture of a medicament for the prevention and treatment of fibrosis, and more preferably for the prevention and treatment of fibrosis of the peritoneal membrane. In a preferred embodiment, the steroid hormone is a gliucocorticoid, and more preferably is the hydrocortisone.

The composition could be a culture media. Then, another aspect of the invention relates to a culture media, hereinafter culture media of the invention, comprising a steroid hormone, preferably a glucocorticoid, and more preferably the hydrocortisone, and low concentration of serum with addition of culture supplements B27 and beta-mercaptoethanol (antioxidant). Another aspect of the invention relates to the use of the culture media of the invention to avoid epithelial-mesenchymal transition, and preferably to form cobblestone-like pavement in mesothelial cells cultured on plastic surface as well as on biological scaffold such is the case of anterior lens capsule (collagen lamina), as shown in the examples of the present invention. In a preferred embodiment, the steroid hormone is a gliucocorticoid, and more preferably is the hydrocortisone.

Along the description and claims, the word “comprises” and variants thereof do not intend to exclude other technical features, supplements, components or steps. For persons skilled in the art, other objects, advantages and features of the invention will be understood in part from the description and in part from the practice of the invention. The following examples and drawings are provided by way of illustration and they are not meant to limit the present invention.

### EXAMPLES OF THE INVENTION

The following specific examples provided in this patent document serve to illustrate the nature of the present invention. These examples are included only for illustrative purposes and must not be interpreted as limiting to the invention which claimed herein. Therefore, the examples described below illustrate the invention without limiting the field of application thereof.

### Example 1

### Materials and Methods

Enzymatic Isolation of Adipose Tissue Mesothelial Cells

Adult Mesothelial cells were isolated from the visceral adipose tissue of adult mouse female (strain CD1). A surgical isolation of visceral uterine fat pads was performed with the use of fine scissors and scalpel (n=7). Adipose fat pads isolated from 2 mice were washed in sterile phosphate buffer saline or PBS (10010-015, Gibco) and transferred in an enzymatic solution (10 ml) consisting of PBS containing 0.25% trypsin (15400-054, Gibco) and 2% bovine serum albumin or BSA (A4503, Sigma Aldrich, Saint Louis, Mo., USA). Tubes were then transferred into a water bath at 37° C. during 20 minutes. Tubes were regularly and gently shaken during all the trypsinization period to improve the contact of trypsin with adipose fat pads and mesothelial cells detachment, Tubes were then put straight in a rack to allow the complete swim up of digested fat pads. The underlying trypsin solution containing released mesothelial cells was then gently collected and centrifuged (7 minutes, 1500 rpm). The resulting pellet was then resuspended into 2 ml Red Blood Cell Lysis Buffer (R7757, Sigma-Aldrich) and gently mixed during 1 minute, After erythrocytes lysis, cells were centrifuged again (7 minutes, 1500 rpm) and finally resuspended in 2 ml PBS containing 2% BSA. Concentration in viable ATMCs was determined with the use of a Neubaeur haemocytometer and the trypan blue exclusion test.

Cultivation of Adipose Tissue Mesothelial Cells in MRPM

A total number of 3.5×10⁵ ATMCs was seeded in each well of a 6 wells culture dishes (140685, Nunclon™ Δ Surface) into 1.5 ml of a culture media that we termed MRPM (Mesothelial Retaining Phenotype Media). The formulation of MRPM was found to be optimial to inhibit epithelial-to-mesenchymal transition (EMT) of ATMCs induced by provasculogenic growth factors (EGF, TGF-β1, FGF and PDGF-BB) (J Am Soc Nephrol. 2011 September; 22(9):1682-95; Dev Dyn 236: 2973-2979) which are components of the fetal bovine serum (FBS). MRPM was formulated from a DMEM low glucose GlutaMax media (21885-05, Gibco) supplemented with a low concentration (2%) of inactivated FBS (Lonza), 1% B27 supplements (17504, Gibco), 1% penicillin-streptomycin (Gibco), 100 μM β-mercaptoethanol (31350-010, Gibco) and a high concentration (1 μg/ml) of the glucocorticoid hydrocortisone (Sigma Aldrich) to inhibit EMT (Lab Invest. 2013 February; 93(2):194-206), Mesothelial cells were cultured for a duration of 2 days in an incubator (5% CO₂, 37° C.) and were finally harvested with 0.05% trypsin (15400-054, Gibco). Concentration in viable ATMCs was assessed with a Neubaeur haemocytometer by using the trypan blue exclusion test and was adjusted to a final concentration of 1.6×10⁶ cells/ml. ATMCs were then seeded on top of the decellularized basal membrane of Anterior Lens Capsules (HALCs), Aliquots of freshly isolated ATMCs were also cultured in similar conditions (48 hours in MRPM) into hydrophilic μ-Dish (45079, Ibidi GmbH, Germany) to characterize their immunophenotype by immunofluorescence against β-catenin and ZO-1 (FIG. 1).

Anterior Lens Capsules

Positioning of Anterior Lens Capsules

A total of 34 Anterior Lens Capsules (HALCs) were obtained during normal cataract surgery procedures. In 6 cases the capsulorhexis was done with a femtosecond laser system achieving a size of 4.5 mm. In the rest of the cases manual capsulorhexis was done targeting a 5 mm diameter. All the capsules were stored in distilled water (MARCA) in order to accomplish a total decellularized basal membrane. To note, HALCs stored in distilled water get invariantly rolled or folded due to their natural convex structure. Trypan blue staining of folded HALCs (n=7) was performed to identify their side corresponding to their decellularized basal membrane. All HALCsexamined presented their outermost external side was invariantly stained with trypan blue indicating thus that it corresponded to their decellularized basal membrane.

HALCs stored into sterile distilled water were gently collected with the help a of a sterile 21 gauge needle and transferred into cell culture dishes of diameter 35 mm vented with 4 inner rings (627170, Greiner CELLSTAR®). A single HALC was deposited in each ring in a reduced volume of 100 μl distilled water (MARCA). HALCs were then correctly oriented under magnification with the help of fine tweezers. The complete flattening of HALCs onto the plastic surface was gradually achieved by eliminating gradually water with the use of a pipette and of sterile 21 gauge needles.

Seeding of ATMCs onto Basal Membrane of HALCs

A volume of 60 μl of MRPM containing 10⁵ ATMCs was then carefully dropped onto the decellularized basal membrane of the HALCs. Cell culture dishes were then placed into a 100 mm Petri dish (172958, Nunclon™ ΔSurface, Nunc) containing sterile tissue soaked in distilled water to maintain optimal humidification and finally transferred in the incubator at (5% CO₂, 37° C.). After 2 hours of incubation, the majority of ATMCs (80-90%) were already adhered on top of HALCs basal membrane. An additional volume of 120 μl of MRPM was then carefully added at the border of the rings and cultures transferred in the incubator for other 4 hours of culture. After this time, a final volume of 1.2 ml of MRPM was carefully added out of the rings and then to the rings to maintain the cultures with sufficient volume of culture media until the next day. MRPM was then exchanged after 24 (1.5 ml) and 48 hours (1.5 ml) of culture. A culture period of 72 hours was found to be sufficient y optimal to obtain a complete cellular coverage of the HALCs basal membrane.

Methods of Evaluation

Optic Microscope

HALCs cultured for 72 hours in MRPM were fixed in cold PBS containing 4% paraformaldehyde or PFA (P6148, Sigma Aldrich) for 20 minutes. HALCs were then preserved into sterile PBS at 4° C. until analysis. Phase contrast images of HALCs were taken with a microscope Olympus IX71 equipped with the digital image processing softwares DPController and DPManager (Olympus.www.olympus.co.uk).

Immunofluorescence

The mesothelial phenotype of ATMCs cultured for 48 hours in MRPM into hydrophilic p-Dish as well as mesothelialized HALC cultures was analyzed with immunofluorescence. ATMCs were fixed in PBS containing 4% PFA during 20 minutes at 4° C. ATMCs were incubated overnight at 4° C. in a permeabilizing and blocking solution consisting of PBS supplemented with 0.5% Triton X-100 (T8787, Sigma Aldrich) and 3% BSA. Antibodies were diluted in permeabilizing and blocking solution and incubated for 1 hour at 4° C. Primary and conjugated antibodies used in this study are described in Table 1 (see below). Secondary antibodies used in this study were a goat anti-mouse IgG-Alexa Fluor 488 nm (A11029, InVitrogen) and a goat anti-rabbit-Alexa Fluor 488 nm (A11034, InVitrogen). Secondary antibodies were diluted at 1/300 and incubated for 30 minutes at RT. Nuclei were counterstained with 1 μg/ml bisBenzimide H 33342 trihydrochloride (Hoechst 33342). Fluorescence images were captured with an inverted fluorescence microscope Olympus IX71 as described above.

TABLE 1
List of primary and conjugated antibodies used in the study

| Antibody | Supplier | Reference | Species |
|---|---|---|---|
| Primary antibodies | | | |
| β-catenin | Cell Signalling | 9581 | Rabbit |
| ZO-1 | InVitrogen | 40-2200 | Rabbit |
| α-SMA | Sigma | A 5228 | Mouse |
| β-actin | Sigma | A5441 | Mouse |
| Conjugated antibodies | | | |
| F-actin-AF633(phalloidin) | InVitrogen | A22284 | Rat |

Abbreviations:
ZO-1, Zona occludens-1;
α-SMA, alpha-smooth muscle actin;
AF633, Alexa Fluor 633 nm;
PFA, paraformaldehyde

Transmission Electronic Microscope

HALCs seeded with ATMCs and cultured for 72 hours in MRPM were fixed with 1.6% glutaraldehyde in 0.15M sodium cacodylated buffer, pH 7.3 for 1 hour at 4° C. HALCs were post fixed in 1% OsO4 in the same buffer, dehydrated in ethanol and embedded in epoxy resin. Thin sections were cut with a diamond knife, stained with uranyl acetate and lead citrate and examined with a PHILIPS CM-10 transmission electron microscope operating at 80 kV. The electron microscopy data were collected after identification of transversal cuts of mesothelialized HALCs at primary magnification 5,000× and pictures were taken at enlargements of 20,000×.

Quantification of Fluorescence with MetaMorph

Quantification of α-SMA immunofluorescence expression was performed with the use of the Meta Imaging Software MetaMorph Offline version 7.5.1.0 (MDS Analytical technologies, USA). For each immunofluorescence image, were calculated the average values of specific Hoechst 33342 and Alexa 488 nm fluorescence emitted by the cells. Specific fluorescence was obtained after eliminating the average values of background fluorescence emitted by 3 areas lacking cells. Hoechst 33342 and Alexa 488 nm fluorescence were further divided between each other to obtain a relative index of marker expression (arbitrary units). Results are represented as median marker expression ±SEM as deduced from immunofluorescence labellings performed in 3 independent cultures.

Western Blot

Protein lysates (20 μg) were subjected to electrophoresis separation by 10% SDS-PAGE and transferred to a polyvinylidene difluoride membrane (Hybond-P, Amersham, Buckinghamshire, UK). Membranes were blocked in Tris-buffered saline with 2% BSA and 0.2% Tween 20. Blots were incubated overnight with primary antibodies against α-SMA ( 1/1000) and β-actin ( 1/500) and immunoreactive bands were detected by horseradish peroxidise-conjugated secondary antibodies followed by ECL Plus detection system (Amersham).

Obtention of Natural Biomaterials

Natural biomaterials such as silk, obtained from worms and insects, is composed of a filament core protein (fibroin) and a glue-like globular protein (sericin). Due to biocompatibility, slow degradability and remarkable mechanical properties and also the ability to modify its molecular structure and morphology silk proteins are excellent candidates for biomedical applications, specially in tissue-engineering applications. Silk fibroin may be used in various formats (films, fibers, nets, meshes, membranes, yarns, and sponges) to support mesothelial cell adhesion, proliferation, and differentiation in vitro and promote tissue repair in vivo. Cell-based tissue engineering using 3D silk fibroin scaffolds has expanded the use of silk-based biomaterials as promising scaffolds for surrogates of bone, ligament, and cartilage, as well as connective tissues like skin.

To generate 3D porous scaffolds from silk fibroin solution either an all aqueous process or an organic solvent process may be used. Further salt leaching, gas foaming and freeze drying work as methods to generate the interconnected pore structures in the 3D matrices. Salt leaching allows highly porous scaffolds to be formed. A combination of high compressive strength result in pores with controllable pore size and size distribution Silk fibroin generally undergoes a structural transition from random coil to β-sheet structures. Scaffolds with controllable morphological and structural features to match functional needs and degradation rate may be engineered.

Collagen, the primary structural protein in the body, is particularly resistant to proteases. In addition to autologous collagen those tissues rich in fibrous collagen such as skin and tendon are generally used as starting materials to generate collagen for use in drug delivery systems, implants and wound dressings. There are different sources such as, human placenta, xeno origin (porcine and sheep collagen varieties) marine sources and even and recombinant human collagen from transgenic animals. The presence of covalent crosslinks between molecules are major impediment to dissolution of collagen type I from tissue. However in young animals and placenta crosslinking is sufficiently low to extract a few percent under appropriate conditions.

Neutral salt solution (0.15-2 M NaCl) or dilute acetic acid will extract freshly synthesized and negligibly crosslinked collagen molecules present in the tissue. The extracted material is purified by dialysis, precipitation, and centrifugation. Dilute acidic solvents, e.g. 0.5 M acetic acid, citrate buffer, or hydrochloric acid pH 2-3 are more efficient than neutral salt solutions. However, dilute acids will not disassociate less labile crosslinks such as keto-imine bonds. Additional collagen material can be solubilized by an aqueous solution comprised of alkali hydroxide and alkali sulfate, e.g. approximately 10% sodium hydroxide and 10% sodium sulfate for approximately 48 h.

Results

Characterization of ATMCs Cultures in RMPM.

ATMCs were detached by trypsinization of the mesothelium covering of the murine uterine adipose tissue (FIG. 1A). ATMCs were mainly detached in the form of small irregular sheets of flattened cells (not shown). After few minutes, these sheets adopted grap-like morphologies as a result of their full retraction. The cultivation of ATMCs for 48 hours in is MRPM formulation generated monolayers displaying the typical cobblestone morphology of squamous epithelial cells. The majority of ATMCs display flattened round and polygonal epitheloid morphologies. Some cells were small refringent cells corresponded to ATMCs undergoing proliferation as also indicated by Ki-67 nuclear expression staining. According to their mesothelial phenotype, ATMCs displayed typical intercellular expression of the epithelial junction proteins β-catenin and ZO-1. E-cadherin was expressed in lower extent at intercellular contact between cells. Furthermore, ATMCs also displayed strong nuclear expression of Wilm's Tumor Protein (WT1), a transcription factor strongly expressed in embryonic mesothelial cells and adult mesothelial cells subjected to in vitro culture in serum containing media⁴⁹. Consistent with their mesothelial phenotype, MRPM cultured ATMCs also display membrane surface expression of the mesothelial protein mesothelin. MRPM accordingly displayed F-actin ring-like staining confined to their inner cell membrane and lacked F-actin and alpha smooth muscle actin positive myofibrils that developed when ATMCs undergo epithelial-to-mesenchymal transition. The MRPM cultured ATMCs were confirmed to lack expression of the pan-endothelial marker CD31. Around 30-40% of ATMCs were judged to undergo proliferation as indicated by their nuclear immunofluorescence of Ki-67.

Anterior Lens Capsule description.

Anterior Lens Capsules (HALCs) obtained by capsulorhexis were transparent sheets of around 5 millimeters diameter and 20 μm thickness. Transmission Electronic Microscope (TEM) analysis of these decellularized HALCs could confirm that their basal membrane surface was exempt of epithelial cells, indicating that their decellularization was fully accomplished within sterile water. Only few rest of small round cellular debris could be observed in few areas (data not shown). Accordingly, these cellular debris were detected by small blue dots in HALCs counterstained with trypan blue (data not shown). These blue dots were invariantly observed of the external side of convex HALCs, the side corresponding to their decellularized basal membrane (data not shown).

Morphology

Seeding of HALCs basal membrane surface with ATMCs resulted successful. At time of 1-2 hours was sufficient for ATMCs adherence on top of HALCs basal membrane side (data not shown).

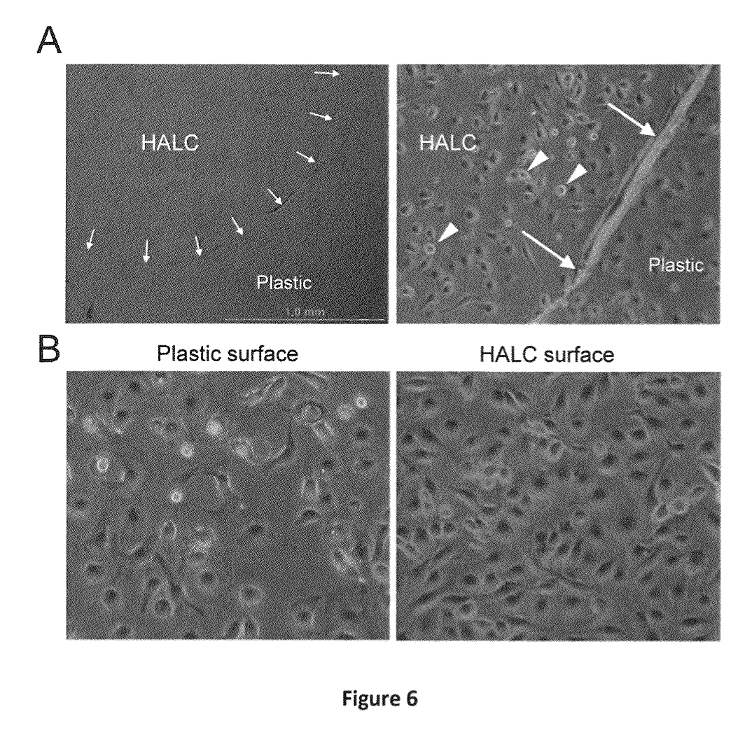

Examination of the seeded HALCs after 12 hours of culture in MRPM indicates that ATMCs could successfully adhere on top of HALCs (FIG. 5). ATMCs cultured for 24 hours on top of HALCs display heterogeneous morphologies, with part of the cells exhibiting proliferation (FIG. 6). Around half of the cells examined were small round and refringent cells, the typical morphology adopted by the pool of ATMCs undergoing proliferation in vitro (data not shown). Other subset of cells exhibited typical flattened and polygonal morphologies of mature squamous mesothelial cells.

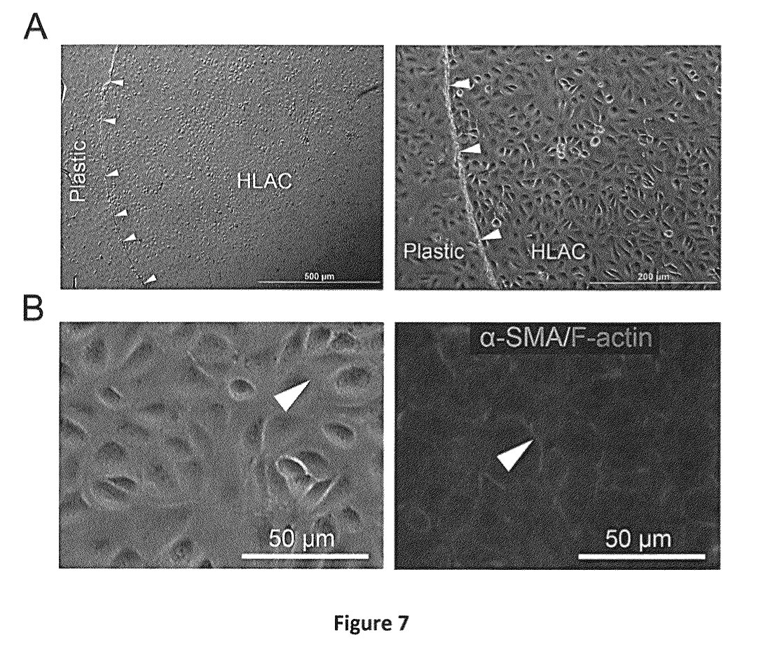

Interestingly, ATMCs cultured for 72 hours on top of HALCs basal membrane displayed increased coverage of their surface (FIG. 7A) when compared to HALCs cultured for only 24 hours (data not shown). In fact, most of the HALCs examined after 72 hours of culture had their entire area fully covered by ATMCs. This finding is of critical relevance, since it indicates that ATMCs could actively proliferate and form a compact monolayer of cells. Interestingly, the monolayer covering HALCs after 72 hours of culture exhibited very few small round or polygonal refringent cells, indicating hence that ATMCs retained contact inhibition growth.

Immunofluorescence

Co-immunofluorescence staining of F-actin and α-SMA was also performed to evaluate whether some cells had undergo EMT (FIG. 7B). Interestingly, ATMCs displayed F-actin expression in their inner cytoplasmic membrane (ring-like staining), a staining pattern that is usually observed in cultured epithelial cells or squamous epithelium. Of particular relevance, ATMCs lacked stress fibers marked with α-SMA and F-actin, indicating thus they were not undergoing EMT. Therefore the morphologic and marker characteristics displayed by ATMCs cultured onto HALCs clearly indicate that ATMCs retained epithelial-like characteristics.

Transmission Electronic Microscope (TEM)

Ultrastructural Characteristics of Mesothelialized HALCs

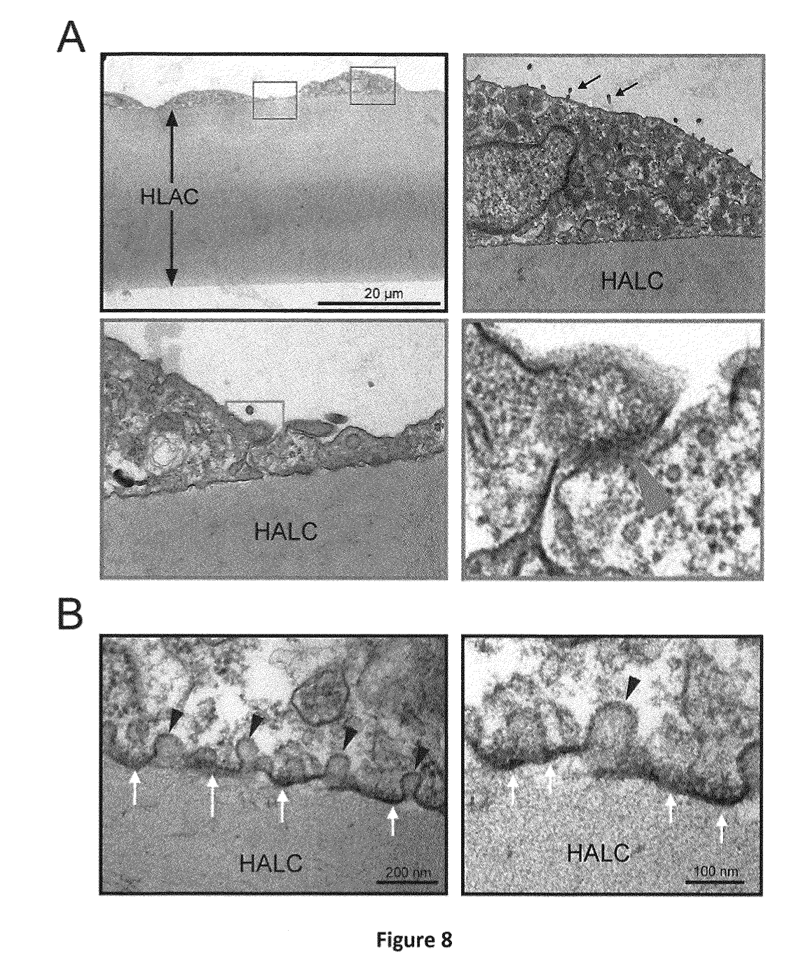

Thin transversal sections of ATMCs covering HALCs (72 h after seeding) were analyzed by Transmission Electronic Microscopy (TEM) (FIG. 8A). Magnification of thin transversal sections of mesothelialized HALCs was performed. Consistent with the retention of their original mesothelial phenotype, ATMCs cultured for 72 hours onto HALCs basal membrane were found to display numerous long protrusions of their apical membrane that correspond to microvilli. Furthermore, the majority of ATMCs examined displayed numerous mitochondria and abundant rough endoplasmic reticulum (RER), two cytoplasmic organelles that are particularly abundant in mesothelial cells.

Adhesions: Cell-Cell and Cell-Basal Membrane

Consistent with their retention of mesothelial phenotype, ATMCs displayed dense-electron tight junction complexes at apicolateral cell-cell contact (FIG. 8B). The basal membrane of ATMCs was found to be in close contact with HALCs surface. Interestingly, the basal membrane of ATMCs displayed numerous invaginations (FIG. 8B). Intercalated between these invaginations, other segments of the basal membrane were thicker and strongly electron-dense, featuring the characteristics of tight junction adhesion complexes.

### Example 2

### Material and Methods

Isolation of Mouse ATMCs

ATMCs were isolated from CD1 adult female mice. Guidelines for the animal research protocols were established and approved by the Animal Experimentation and Ethics Committee of CABIMER. Briefly, uterine cords and adipose tissue were surgically separated. Enzymatic detachment of ATMCs from fat pads was performed with minor modifications as previously reported for the isolation of mouse uterine MCs51. [Lachaud CC, Pezzolla D, Dominguez-Rodriguez A, Smani T, Soria B, Hmadcha A. Functional vascular smooth muscle-like cells derived from adult mouse uterine mesothelial cells. PLoS One. 2013; 8(2):e55181]

Culture of ATMCs

ATMCs were seeded (35000 cells/cm2) into T-25 flask (136196, Nunc) into 5 ml of a Mesothelial Retaining Phenotype Media (MRPM) consisting of a DMEM low glucose GlutaMax media (21885-05, Gibco) supplemented with 2% FBS (Lonza), 1% B27 supplements (17504, Gibco), 1% penicillin-streptomycin (Gibco), 100 μM β-mercaptoethanol (31350-010, Gibco) and 1 μg/ml of hydrocortisone (Sigma Aldrich). ATMCs were cultured for 2 days in MRPM (5% CO2, 21% 02 and 37° C.) and harvested with trypsin (15400-054, Gibco).

Murine Corneal Endothelial Cells Culture

Primary murine corneal endothelial cells (MCECs) were isolated by collecting corneas from two to three adult CD1 mice (2-6 months old). Corneal endothelium layer was gently stripped off with the help of a scalpel. MCECs clusters released through stripping were then subjected to primary explant culture during 7 days in an incubator (5% CO2, 21% 02, 37° C.) in a medium consisting of a DMEM low glucose GlutaMax media (21885-05, Gibco) containing 5% FBS (Lonza), 1% penicillin-streptomycin (Gibco), 1% non-essential amino acids (Gibco), 100 μM β-mercaptoethanol (31350-010, Gibco), 1×ITS (41400045, Gibco), 10 ng/ml recombinant murine bFGF (450-33, PeproTech), 10 ng/ml recombinant murine EGF (315-09, PeproTech) and 1 μg/ml hydrocortisone (Sigma Aldrich). Primary expanded MCECs were further subcultured three times at 5000 cells/cm2 during 4-5 days into T-75 flasks (156499, Nunc). MCECs in subculture passage 3 used in the study were termed P3 MCECs.

Immunofluorescence

For immunofluorescence, cells were cultured into μ-Dish (45079, Ibidi GmbH, Germany). For cell-surface antigens detection, cells were fixed with 4% PEA (P6148, Sigma Aldrich) and blocked in PBS containing 3% BSA (PBS-BSA). For intracellular antigens detection, cells were permeabilized with 0.5% Triton X-100 (Sigma, T8787) or cold methanol (−20° C.) and then blocked into PBS-BSA. Antibodies used in this study are described in supporting information Table S1. Nuclei were counterstained with 1 μg/ml Hoechst 33342 (Sigma, 14533). Fluorescence images were captured with an inverted fluorescence microscope Olympus IX71 (Olympus. www.olympus.co.uk).

Quantitative Reverse Transcription-Polymerase Chain Reaction

Total RNA content was extracted with RNeasy Mini Kit (74104, QIAGEN), and reverse-transcribed into cDNA by using MMLV reverse transcriptase (Promega, Madison, Wis., USA)). Quantitative real-time PCR was performed using SYBR-Green and detected using an ABI Prism 7500 system (Applied Biosystems, Foster City, Calif., USA). Gene expression was normalized to HYWAZ mRNA (TATAA Reference Gene Panel, ref. D101-D136, TATAA Biocenter AB, Göteborg, Sweden), Stripped corneal endothelium served as the calibrator sample. Primers sequences are in listed in supporting information Table S2.

TABLE S2
List of primers used
for quantitative RT-PCR

| Gene | Forward | Reverse | pb |
|---|---|---|---|
| COL4A2 (Collagen | TAACACCCACC | CAGCTATTGT | 100 |
| alpha-2(IV)chain) | TTGGAGAGC | TGGGGACACC | |
| COL8A2 (Collagen | CCTGGAGTGAT | AGGGAAACGG | 139 |
| alpha-2(VIII)chain) | GTTGTGAGC | AAGAGTGACC | |
| SCL4A4 (Na⁺, HCO₃⁻ | GGCTTTGCTAG | AGGAGCATAC | 143 |
| cotransporter 1) | TCACCATCC | CACCATGAGG | |
| CAR2 (Carbonic | CCATTAAAACA | GTCACACATT | 127 |
| anhydrase II) | AAGGGGAAGC | CCAGCAGAGG | |
| ATP1A1 (Na⁺/ | AGTGGACCTAC | TCTTGCAGAT | 102 |
| K⁺-ATPase αl) | GAGCAGAGG | GACCAAGTCG | |
| CDH2 (N-cadherin) | AGGACCCTTTC | ATAATGAAGA | 117 |
| | CTCAAGAGC | TGCCCGTTGG | |

Human Anterior Lens Capsules

Adjustment of HALCs

A total of 34 human anterior lens capsules (HALCs) of 5 mm diameter were obtained after patient informed consent during normal cataract surgery procedures. HALCs were directly stored in distilled water to accomplish a total decellularized basal membrane. To note, HALCs were invariantly rolled or folded due to their natural convex structure, being their inner side corresponding to the decellularized epithelial side. HALCs were further distributed into 35 mm cell culture dishes vented with 4 inner rings (627170, Greiner CELLSTAR®) into 100 μl of distilled water. HALCs were correctly oriented with their outer side facing plastic surface. Their complete flattening was achieved by eliminating gradually water with the use of a pipette and needles.

Seeding of HALCs with ATMCs

An initial volume of 60 μl MRPM containing 105 ATMCs was dropped onto HALCs. Culture dishes containing the seeded HALCs were then placed in a 140 mm Petri dish containing pieces of tissue paper soaked in water to maintain optimal humidification and were finally transferred in an incubator (5% CO2, 21% 02, 37° C.). Around 80-90% of ATMCs were already firmly adhered after 2 hours of culture. An additional volume of 120 μl of MRPM was then carefully added to the rings. After 6 hours, a final volume of 1.2 ml of MRPM was carefully added out of the rings. After 24 and 48 hours, MRPM (1.5 ml) was exchanged. After 72 hours, HALCs typically displayed complete cellular coverage and termed ATMCs-HALCs composites.

Transmission Electronic Microscope

ATMCs-HALCs composites n=8) were fixed with 1.6% glutaraldehyde in 0.15M sodium cacodylated buffer (pH 7.3) for 1 hour at 4° C. HALCs were then post fixed in 1% OsO4 in the same buffer, dehydrated in ethanol and embedded in epoxy resin. Thin sections were cut and stained with uranyl acetate and lead citrate to be examined with a PHILIPS CM-10 transmission electron microscope operating at 80 kV. The electron microscopy data were collected after identification of transversal cuts of ATMCs-HALCs composites at primary magnification 5,000× and pictures were taken at enlargements of 20,000×.

Immunohistochemistry

Mouse corneas from CD1 adult mice were fixed with 4% PFA, permeabilized and blocked into PBS-Tx-BSA and finally embedded in OCT cryostat sectioning medium. Alternatively, corneas were fixed and permeabilized in cold methanol for Na+/K+-ATPase and β-catenin detection. Thin sections (15 μm) were mounted onto poly-L-lysine coated glass slides and incubated with primary and secondary antibodies (supporting information Table S1).

TABLE S1
List of antibodies used in immunofluorescence study

| | Supplier | Reference | Clone | Source | Fixation | IF |
|---|---|---|---|---|---|---|
| Primary antibodies | | | | | | |
| β-catenin | C.Signalling | 9581 | polyclonal | Rbt | MeOH | 1/200 |
| Zona Occludens 1 (ZO-1) | InVitrogen | 40-2200 | polyclonal | Rbt | PFA | 1/200 |
| α-smooth muscle actin (α-SMA) | Sigma | A 5228 | 1A4 | M | MeOH | 1/300 |
| Collagen alpha-2(VIII)chain | SCBT | Sc-134947 | H-45 | Rbt | PFA | 1/100 |
| (COL8A2) | | | | | | |
| N-cadherin (CDH2) | Abcam | Ab76057 | — | Rbt | PFA | 1/100 |
| Na⁺/K⁺ ATPase α1 (ATP1A1) | SCBT | Sc-21712 | C464.4 | Ms | MeOH | 1/100 |
| Na⁺, HCO₃⁻ cotransporter 1 | SCBT | Sc-162214 | — | G | MeOH | 1/100 |
| (SCL4A4) | | | | | | |
| Conjugated antibodies | | | | | | |
| Alexa Fluor ® 633 Phalloidin (F- | InVitrogen | A22284 | — | Rt | MeOH | 1/50 |
| actin) | | | | | | |

| Secondary antibodies | Supplier | Reference | Label | Source | IF |
|---|---|---|---|---|---|
| Mouse IgG | InVitrogen | A11029 | AF 488 nm | G | 1/300 |
| Rabbit IgG | InVitrogen | A11034 | AF 488 nm | G | 1/300 |
| Goat IgG | InVitrogen | A11078 | AF 488 nm | R | 1/300 |

Abbreviations:
BD; Becton Dickinson,
SCBT; Santa Cruz Biotechnology,
PE; Phycoerythrin,
AF; Alexa Fluor,
PFA; paraformaldehyde,
MeOH; metanol,
G; goat,
Rbt; rabbit,
Hst, hamster;
Ms; mouse,
Rt; rat.

Statistical Analysis

Values are presented as mean±SD. Statistical significance was calculated by using an unpaired Student's t-test, p<0.05 was considered significantly different.

1. A composition comprising mesothelial cells for use in the treatment of a diseased or damaged tissue or organ.

2. The composition for use according to claim 1, wherein the mesothelial cells are autologous adipose tissue mesothelial cells (ATMCs).

3. The composition for use according to any one of claims 1-2, wherein the mesothelial cells are cultivated in a suitable medium further comprising a glucocorticoid

4. The composition for use according to any one of claims 1-2, wherein the mesothelial cells are cultivated in Mesothelial Retaining Phenotype Media (MRPM) comprising a glucocorticoid wherein said Mesothelial Retaining Phenotype Media (MRPM) consists of a DMEM low glucose media supplemented with a low concentration (2%) of inactivated fetal bovine serum, 1% B27 supplements, 1% penicillin-streptomycin and 100 μM of the anti oxidant β-mercaptoethanol.

5. The composition for use according to claim 3 or 4, wherein the glucocorticoid is hydrocortisone at a concentration of 0.1 to 100 μg/ml.

6. The composition for use according to any one of claims 1-5, wherein the diseased or damaged tissue is the endothelium.

7. The composition for use according to claim 6, wherein the diseased or damaged tissue is selected from the endothelium that lines the interior surface of blood vessels and lymphatic vessels, or the corneal endothelium.

8. The composition for use according to any one of claims 1-5, wherein the diseased or damaged tissue is a serous membrane.

9. An in vitro method for preparing an artificial tissue comprising;
a. sedding a support material with isolated mesothelial cells, and
b. Culturing the isolated mesothelial cells in the support material of (a) in a suitable medium comprising a glucocorticoid.

10. An in vitro method for preparing an artificial tissue comprising the following steps:
a. obtaining a composition comprising isolated mesothelial cells;
b. culturing the isolated mesothelial cell composition of (a) in a suitable medium comprising a glucocorticoid;
c. seeding the cultured isolated mesothelial cells from (b) onto a support material, and
d. culturing the isolated mesothelial cells on the support material of (c) in a suitable medium comprising a glucocorticoid.

11. An in vitro method for preparing an artificial tissue comprising the following steps:
a. adding a composition comprising trypsin to a sample of tissue comprising mesothelial cells,
b. culturing the isolated mesothelial cell composition of (a) in a suitable medium comprising a glucocorticoid;
c. seeding the cultured isolated mesothelial cells from (b) onto a support material, and
d. culturing the isolated mesothelial cells on the support material of (c) in a suitable medium comprising a glucocorticoid.

12. The in vitro method according to any one of claims 9-11, wherein the suitable medium is the Mesothelial Retaining Phenotype Media (MRPM) comprising a glucocorticoid wherein said Mesothelial Retaining Phenotype Media (MRPM) consists of a DMEM low glucose media supplemented with a low concentration (2%) of inactivated fetal bovine serum, 1% B27 supplements, 1% penicillin-streptomycin and 100 μM of the anti oxidant β-mercaptoethanol.

13. The in vitro method according to any one of claims 9-12, wherein the glucocorticoid is hydrocortisone.

14. The in vitro method according to claim 14, wherein the concentration of the hydrocortisone ranges from 0.1 to 100 μg/ml.

15. The in vitro method according to claim 13, wherein the concentration of the hydrocortisone is 1 μg/ml

16. The in vitro method according to any one of claims 9-15, wherein the mesothelial cells are derived from adipose tissue.

17. The in vitro method according to claim 16, wherein the mesothelial cells are autologous adipose tissue mesothelial cells (ATMCs).

18. The in vitro method according to any one of claims 9-17, wherein the support material is from natural or synthetic origin.

19. The in vitro method according to claim 18, wherein the support material from natural origin is selected from the list consisting of: silk, decellularized bovine serosal membranes isolated from the mesentery, decellularized bovine pericardium and combinations thereof.

20. The in vitro method according to any one of claims 9-17, wherein the support material is threads with a monofilament or multifilament structure.

21. The in vitro method according to claims 9-17, wherein the support material is a silk nanofibers lamina.

22. The in vitro method according to any one of claims 9-17, wherein the support material is a suturing thread joined to a needle.

23. The in vitro method according to any one of claims 9-17, wherein the support material is a staple.

24. An artificial tissue obtainable by the method according to any one of claims 9-23.

25. Use of the artificial tissue according to claim 24, for evaluating a pharmacological and/or chemical product.

26. The artificial tissue of claim 24 for use in medicine or for use as a medicament.

27. The artificial tissue of claim 24, for use in the treatment of a diseased or damaged tissue or organ.

28. The artificial tissue for use according to claim 27, wherein the diseased or damaged tissue is the endothelium.

29. The artificial tissue for use according to claim 28, wherein the diseased or damaged tissue is selected from the endothelium that lines the interior surface of blood vessels and lymphatic vessels, or the corneal endothelium.

30. The artificial tissue for use according to 27, wherein the diseased or damaged tissue is corneal endothelium.

31. The artificial tissue for use according to claim 27, wherein the diseased or damaged tissue is a serous membrane.

32. The artificial tissue for use according to claim 27, wherein the diseased or damaged tissue is a simple squamous epithelium with all the cells anchored to the basement membrane.

33. The artificial tissue for use according to claim 27, wherein the diseased or damaged tissue is the synovial membrane.

34. The artificial tissue for use according to claim 27, wherein the diseased or damaged tissue is the inner tracheal layer.

35. The artificial tissue for use according to claim 27, wherein the diseased or damaged tissue is the esophageous epithelium.

36. The artificial tissue for use according to claim 27, wherein the diseased or damaged tissue is the bladder urothelium.

37. A pharmaceutical composition comprising a mesothelial cell or the artificial tissue according to claim 24.

38. The pharmaceutical composition according to claim 37, further comprising a pharmaceutically acceptable carrier.

39. The pharmaceutical composition according to any of claims 37-38, further comprising a further active ingredient.
